SEQUENCE LISTING (56 sequences)

SEQ ID NO: 1 - protein, 394 aa, Homo sapiens
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 2 - protein, 394 aa, Homo sapiens
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAEFLEAIPLSIPPEVKFNKPFVFLMIEQNTKSPLFMGKVVNPTQK

SEQ ID NO: 3 - protein, 25 aa, Homo sapiens
GTEAAGAMFLEAIPMSIPPEVKFNK

SEQ ID NO: 4 - protein, 25 aa, Homo sapiens
GTEAAGAEFLEAIPLSIPPEVKFNK

SEQ ID NO: 5 - protein, 25 aa, Homo sapiens
GTEAAGALFLEAIPLSIPPEVKFNK

SEQ ID NO: 6 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 217 aa, Artificial Sequence
  Modified IgG1 Fc polypeptide
APELLGGPSVFLFPPKPKDTLIISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 9 - protein, 227 aa, Artificial Sequence
  Modified IgG1 Fc polypeptide
DKTHTCPPCPAPELLGGPSVFLFPPKPKDTLIISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 10 - protein, 216 aa, Artificial Sequence
  Modified IgG1 Fc polypeptide
APELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 226 aa, Artificial Sequence
  Modified IgG1 Fc polypeptide
DKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 217 aa, Artificial Sequence
  Modified IgG1 Fc polypeptide
APEVAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 227 aa, Artificial Sequence
  Modified IgG1 Fc polypeptide
DKTHTCPPCPAPEVAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 216 aa, Artificial Sequence
  Modified IgG1 Fc polypeptide
APEVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 226 aa, Artificial Sequence
  Modified IgG1 Fc polypeptide
DKTHTCPPCPAPEVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 216 aa, Artificial Sequence
  Modified IgG1 Fc polypeptide
APEVAGPSVFLFPPKPKDTLIISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 17 - protein, 226 aa, Artificial Sequence
  Modified IgG1 Fc polypeptide
DKTHTCPPCPAPEVAGPSVFLFPPKPKDTLIISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 18 - protein, 216 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 19 - protein, 216 aa, Artificial Sequence
  Modified IgG2 Fc polypeptide
APPVAGPSVFLFPPKPKDTLIISRDPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHNHYTQKSLSLSPGK

SEQ ID NO: 20 - protein, 217 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 21 - protein, 217 aa, Artificial Sequence
  Modified IgG3 Fc polypeptide
APELLGGPSVFLFPPKPKDTLIISRDPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVLHEALHNRFTQKSLSLSPGK

SEQ ID NO: 22 - protein, 216 aa, Artificial Sequence
  Modified IgG3 Fc polypeptide
APELLGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 23 - protein, 217 aa, Artificial Sequence
  Modified IgG3 Fc polypeptide
APEVAGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 24 - protein, 216 aa, Artificial Sequence
  Modified IgG3 Fc polypeptide
APEVAGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 25 - protein, 216 aa, Artificial Sequence
  Modified IgG3 Fc polypeptide
APEVAGPSVFLFPPKPKDTLIISRDPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVLHEALHNRFTQKSLSLSPGK

SEQ ID NO: 26 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 217 aa, Artificial Sequence
  Modified IgG4 Fc polypeptide
APEFLGGPSVFLFPPKPKDTLIISRDPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 29 - protein, 229 aa, Artificial Sequence
  Modified IgG4 Fc polypeptide
ESKYGPPCPSCPAPEFLGGPSVFLFPPKPKDTLIISRDPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 30 - protein, 216 aa, Artificial Sequence
  Modified IgG4 Fc polypeptide
APEFLGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 228 aa, Artificial Sequence
  Modified IgG4 Fc polypeptide
ESKYGPPCPSCPAPEFLGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 217 aa, Artificial Sequence
  Modified IgG4 Fc polypeptide
APEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 229 aa, Artificial Sequence
  Modified IgG4 Fc polypeptide
ESKYGPPCPSCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 217 aa, Artificial Sequence
  Modified IgG4 Fc polypeptide
APEVAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 229 aa, Artificial Sequence
  Modified IgG4 Fc polypeptide
ESKYGPPCPSCPAPEVAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 229 aa, Artificial Sequence
  Modified IgG4 Fc polypeptide
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 229 aa, Artificial Sequence
  Modified IgG4 Fc polypeptide
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 229 aa, Artificial Sequence
  Modified IgG4 Fc poplypeptide
ESKYGPPCPPCPAPEFEGGPSVFLFPPKPKDTLYISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - protein, 229 aa, Artificial Sequence
  Modified IgG4 Fc polypeptide
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 40 - protein, 229 aa, Artificial Sequence
  Modified IgG4 Fc polypeptide
ESKYGPPCPPCPAPEFEGGPSVFLFPPKPKDTLYISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 41 - protein, 217 aa, Artificial Sequence
  Modified IgG4 Fc polypeptide
APEFEGGPSVFLFPPKPKDTLIISRDPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 42 - protein, 229 aa, Artificial Sequence
  Modified IgG4 Fc polypeptide
ESKYGPPCPSCPAPEFEGGPSVFLFPPKPKDTLIISRDPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 43 - protein, 229 aa, Artificial Sequence
  Modified IgG4 Fc polypeptide
ESKYGPPCPPCPAPEFEGGPSVFLFPPKPKDTLIISRDPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 44 - protein, 218 aa, Homo sapiens
IAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKG

SEQ ID NO: 45 - protein, 626 aa, Artificial Sequence
  AAT and IgG-Fc polypeptide fusion
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 622 aa, Artificial Sequence
  AAT and IgG-Fc polypeptide fusion
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 47 - protein, 626 aa, Artificial Sequence
  AAT and IgG-Fc fusion polypeptide
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAEFLEAIPLSIPPEVKFNKPFVFLMIEQNTKSPLFMGKVVNPTQKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 394 aa, Artificial Sequence; identical to SEQ ID NO: 2
  AAT polypeptide

SEQ ID NO: 49 - protein, 622 aa, Artificial Sequence
  AAT and IgG-Fc fusion polypeptide
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAEFLEAIPLSIPPEVKFNKPFVFLMIEQNTKSPLFMGKVVNPTQKERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 50 - protein, 622 aa, Artificial Sequence
  AAT and IgG-Fc fusion polypeptide
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGALFLEAIPLSIPPEVKFNKPFVFLMIEQNTKSPLFMGKVVNPTQKERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 51 - protein, 394 aa, Artificial Sequence
  AAT polypeptide
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGALFLEAIPLSIPPEVKFNKPFVFLMIEQNTKSPLFMGKVVNPTQK

SEQ ID NO: 52 - protein, 1025 aa, Artificial Sequence [sequence omitted]

SEQ ID NO: 53 - protein, 624 aa, Artificial Sequence
  AAT and IgG-Fc fusion polypeptide
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAEFLEAIPLSIPPEVKFNKPFVFLMIEQNTKSPLFMGKVVNPTQKGGGGDKTHTCPPCPAPEVAGPSVFLFPPKPKDTLIISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 54 - protein, 623 aa, Artificial Sequence
  AAT and IgG-Fc fusion polypeptide
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAEFLEAIPLSIPPEVKFNKPFVFLMIEQNTKSPLFMGKVVNPTQKESKYGPPCPPCPAPEFEGGPSVFLFPPKPKDTLIISRDPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 55 - protein, 626 aa, Artificial Sequence
  AAT and IgG-Fc fusion polypeptide
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGALFLEAIPLSIPPEVKFNKPFVFLMIEQNTKSPLFMGKVVNPTQKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 625 aa, Artificial Sequence
  AAT and IgG-Fc fusion polypeptide
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAEFLEAIPLSIPPEVKFNKPFVFLMIEQNTKSPLFMGKVVNPTQKGSESKYGPPCPPCPAPEFEGGPSVFLFPPKPKDTLYISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

(19) United States
(12) Patent Application Publication
(10) Pub. No.: US 2022/0265788 A1
(43) Pub. Date: Aug. 25, 2022

(54) AQUEOUS SOLUTION COMPOSITIONS FOR INCREASING STABILITY OF ENGINEERED DIMERIC PROTEINS

(71) Applicant: Arecor Limited, Saffron Walden (GB)

(72) Inventors: Jan JEZEK, Saffron Walden (GB); David GERRING, Saffron Walden (GB); Joshua CREMIN, Saffron Walden (GB); Jorge PINTO, Saffron Walden (GB); Ashraf AMANULLAH, La Jolla, CA (US); Brian LOBO, La Jolla, CA (US); Bradley HAYES, La Jolla, CA (US)

(21) Appl. No.: 17/674,713

(22) Filed: Feb. 17, 2022

(30) Foreign Application Priority Data
Feb. 17, 2021 (GB) 2102258.7

(51) Int. Cl. A61K 38/57; A61K 47/26; A61K 9/08

(52) U.S. Cl. CPC A61K 38/57; A61K 9/08; A61K 47/26

(57) ABSTRACT

The present application relates to aqueous solution compositions of engineered dimeric proteins comprising monomers that comprise at least one human serpin polypeptide operably linked to a human immunoglobulin Fc polypeptide or a polypeptide that is derived from an immunoglobulin Fc polypeptide, at low buffer concentrations and low ionic strength and containing a neutral amino acid. The aqueous solution compositions increase the stability of the an Fc domain in aqueous solution compositions, and in particular increase the stability of an engineered dimeric protein.

### RELATED APPLICATIONS

This application claims the benefit of Patent Application No. 2102258.7, filed in Great Britain on Feb. 17, 2021, the contents of which are hereby incorporated by reference in their entirety

### INCORPORATION BY REFERENCE OF SEQUENCE LISTING

The contents of the text file named INHI-702_001WO Sequence_Listing.txt which was created on Feb. 16, 2022, and is 148 kilobytes in size, are incorporated herein by reference in their entirety.

### COMMON OWNERSHIP UNDER JOINT RESEARCH AGREEMENT

The subject matter disclosed in this application was developed, and the claimed invention was made by, or on behalf of, one or more parties to a Joint Research Agreement that was in effect on or before the effective filing date of the claimed invention. The parties to the Joint Research Agreement are as follows: Arecor Limited and Inhibrx, Inc.

### FIELD OF INVENTION

This invention relates to aqueous solution compositions of an engineered dimeric protein comprising an Fc domain at low buffer concentrations and low ionic strength and containing a neutral amino acid.

### BACKGROUND

Engineered proteins comprising an Fc domain are widely used in therapy. The Fc domain is the C-terminal region of an antibody that interacts with cell surface receptors called Fc receptors and some proteins of the complement system and thereby activate the immune system. In IgG, IgA and IgD antibody isotypes, the Fc domain is composed of two identical protein chain fragments, each of which is derived from the second and third constant domains of the antibody's heavy chain. In IgM and IgE antibody isotypes, the Fc domain is composed of two identical protein chain fragments, each of which is derived from the second, third and fourth constant domains of the antibody's heavy chain. The molecular weight of an Fc domain may typically be in the range 25-40 kDa, and may be larger where glycosylation is present. A wide range of physiological effects result from the activation of the immune system mediated by antibody Fc domain binding, including cell lysis and degranulation of mast cells, basophils and eosinophils.

A wide range of engineered antibody proteins have been developed, including bispecific and trispecific antibodies. A number of engineered proteins have also been developed wherein the Fc, separated from the Fab parts of an antibody molecule (the parts that confer antigen binding specificity) can serve a purpose different from its physiological purpose, in particular, the purpose of extending the in vivo half-life of the engineered protein. WO2013/003641A2 and WO2016/069574A1 (INHIBRX) disclose engineered dimeric proteins that include a serpin polypeptide or an amino acid sequence that is derived from a serpin.

When formulated as aqueous solutions, proteins can be susceptible to degradation and consequent loss of biological activity while stored. The degradation can be physical in nature, including aggregation, precipitation or gel formation. The degradation can also be chemical in nature, including hydrolytic cleavage, deamidation, cyclic imide formation, aspartate/glutamate isomerization or oxidation.

The rates of the degradation processes increase with increasing temperature, and protein therapeutic molecules are generally more stable at lower temperatures. However, it is often challenging to develop a therapeutic protein product that is stable in liquid form for the duration of the intended shelf-life (typically 24 months), even under refrigeration. In addition, to ensure convenience for patients there is often a need to develop products that are stable at elevated temperatures, such as up to 25° C. or up to 30° C., either for a specific period of time or for their entire shelf-life.

One of the most critical parameters to control the stability of protein therapeutics is pH. Therefore, pH optimization is a key step in formulation development. Many therapeutic proteins are formulated at a selected pH between 4.0-8.5. It is thought to be important to ensure that the pH is maintained at the selected value and pH fluctuations are minimized. Therefore, it has been understood that a certain degree of buffering capacity is needed in the formulation. Larger protein molecules typically have some self-buffering capacity due to the presence of ionisable groups amongst the amino acid side chains of the polypeptide backbone.

The present invention provides compositions that increase the stability of engineered proteins that comprise an Fc domain in aqueous solution compositions, and in particular increase the stability of an engineered dimeric protein wherein each monomer of the dimeric protein comprises at least one human serpin polypeptide operably linked to a human immunoglobulin Fc polypeptide or a polypeptide that is derived from an immunoglobulin Fc polypeptide (“the engineered dimeric protein of the invention”).

### SUMMARY OF THE INVENTION

The present disclosure provides an aqueous solution composition of pH in the range 6.0 to 8.0 comprising: an engineered dimeric protein wherein each monomer of the dimeric protein comprises at least one human serpin polypeptide operably linked to a human immunoglobulin Fc polypeptide or a polypeptide that is derived from an immunoglobulin Fc polypeptide; optionally one or more buffers being substances having at least one ionisable group with a pKₐ in the range 4.0 to 10.0 and which pKₐ is within 2 pH units of the pH of the composition; a neutral amino acid; and an uncharged tonicity modifier; wherein the buffers are present in the composition at a total concentration of 0-10 mM; and wherein the total ionic strength of the composition excluding the contribution of the engineered dimeric protein is less than 30 mM.

In some embodiments of the aqueous solution composition disclosed herein, the human serpin polypeptide is a human alpha-1 antitrypsin (AAT) polypeptide or is derived from a human AAT polypeptide. In some embodiments of the aqueous solution composition disclosed herein, each monomer of the dimeric protein comprises one human serpin polypeptide. In some embodiments of the aqueous solution composition disclosed herein, the human serpin polypeptide has the sequence of SEQ ID NO: 1 or 2. In some embodiments of the aqueous solution composition disclosed herein, the human immunoglobulin Fc polypeptide or a polypeptide that is derived from an immunoglobulin Fc polypeptide is a modified human IgG4 Fc polypeptide. In some embodiments of the aqueous solution composition disclosed herein, the human immunoglobulin Fc polypeptide or a polypeptide that is derived from an immunoglobulin Fc polypeptide is a modified human IgG4 Fc polypeptide and has the sequence of any one of SEQ ID NOs: 28-43. In some embodiments of the aqueous solution composition disclosed herein, each monomer of the dimeric protein has the sequence of SEQ ID No: 56.

In some embodiments of the aqueous solution composition disclosed herein, the protein is present at a concentration of 1-400 mg/ml e.g., 10-200 mg/ml e.g. 20-100 mg/ml e.g. 30-60 mg/ml e.g. about 35 mg/ml or about 50 mg/ml.

In some embodiments of the aqueous solution composition disclosed herein, buffers are present at a total concentration of 0.1-10 mM, such as 0.5-10 mM such as 1-10 mM, such as 1-8 mM, such as 1-6 mM, such as 2-6 mM, such as 2-5 mM e.g. 3-5 mM. In some embodiments, the aqueous solution composition disclosed herein is substantially free of buffers.

In some embodiments of the aqueous solution composition disclosed herein, the buffer comprises ionisable groups with pKa within 1 unit of the pH of the composition.

In some embodiments of the aqueous solution composition disclosed herein, the buffer or buffers is/are selected from the group consisting of citrate, histidine, maleate, sulphite, aspartame, aspartate, glutamate, tartrate, adenine, succinate, ascorbate, benzoate, phenylacetate, gallate, cytosine, p-aminobenzoic acid, sorbate, acetate, propionate, alginate, urate, 2-(N-morpholino)ethanesulphonic acid, bicarbonate, bis(2-hydroxyethyl) iminotris(hydroxymethyl)methane, N-(2-acetamido)-2-iminodiacetic acid, 2-[(2-amino-2-oxoethyl)amino]ethanesulphonic acid, piperazine, N,N′-bis(2-ethanesulphonic acid), phosphate, N,N-bis(2-hydroxyethyl)-2-aminoethanesulphonic acid, 3-[N,N-bis(2-hydroxyethyl)amino]-2-hydroxypropanesulphonic acid, triethanolamine, piperazine-N,N′-bis(2-hydroxypropanesulphonic acid), tris(hydroxymethyl)aminomethane (TRIS), N tris(hydroxymethyl)glycine and N-tris(hydroxymethyl)methyl-3-aminopropanesulphonic acid, and salts thereof, and combinations thereof. In some embodiments of the aqueous solution composition disclosed herein, the buffer is selected from the group consisting of citrate, histidine, maleate, tartrate, benzoate, acetate, bicarbonate, phosphate and tris(hydroxymethyl)aminomethane (TRIS), for example, selected from phosphate and TRIS.

In some embodiments of the aqueous solution composition disclosed herein, the uncharged tonicity modifier is selected from the group consisting of polyols, sugars (e.g. monosaccharides and disaccharides) and sugar alcohols. In some embodiments of the aqueous solution composition disclosed herein, the uncharged tonicity modifier is selected from the group consisting of glycerol, 1,2-propanediol, mannitol, sorbitol, glucose, sucrose, trehalose, PEG300 and PEG400, and in particular is selected from glycerol, mannitol, sucrose and trehalose. In some embodiments, the aqueous solution composition disclosed herein comprises a disaccharide as an uncharged tonicity modifier. In some embodiments, the aqueous solution composition disclosed herein comprises sucrose and/or trehalose as the uncharged tonicity modifier, in particular trehalose.

In some embodiments of the aqueous solution composition disclosed herein, the total concentration of the uncharged tonicity modifier, or combination of more than one tonicity modifier, is 50-1000 mM, such as 200-600 mM, 200-500 mM or wherein the total concentration of the uncharged tonicity modifier, or combination of more than one tonicity modifier, is 50-500 mM, such as 100-400 mM, 150-350 mM, 200-300 mM or about 250 mM.

In some embodiments of the aqueous solution composition disclosed herein, the osmolarity of the composition is 200-500 mOsm/L e.g. about 300 mOsm/L or wherein the osmolarity of the composition is 300-500 mOsm/L e.g. about 400-460 mOsm/L.

In some embodiments, the aqueous solution composition disclosed herein comprises a neutral amino acid selected from glycine, methionine, proline, alanine, valine, leucine, isoleucine, phenylalanine, tyrosine, tryptophan, serine, threonine, asparagine and glutamine. In some embodiments, the neutral amino acid is selected from glycine, methionine and proline. In some embodiments, the aqueous solution composition disclosed herein comprises proline as a neutral amino acid. In some embodiments, the aqueous solution composition disclosed herein comprises glycine as a neutral amino acid. In some embodiments, the aqueous solution composition disclosed herein comprises proline and methionine as neutral amino acids. In some embodiments, the aqueous solution composition disclosed herein comprises glycine and methionine as neutral amino acids.

In some embodiments of the aqueous solution composition disclosed herein, the total concentration of the one or more neutral amino acids in the composition is 20 to 600 mM, such as 20 to 500 mM, such as 20 to 400 mM , such as 20 to 300 mM e.g. 50 to 300 mM. In some embodiments of the aqueous solution composition disclosed herein, the total concentration of the one or more neutral amino acids in the composition is 50 to 200 mM, 100 to 200 mM or 100 to 150 mM.

In some embodiments of the aqueous solution composition disclosed herein, the total ionic strength of the composition excluding the contribution of the engineered dimeric protein is less than 20 mM. In some embodiments of the aqueous solution composition disclosed herein, the total ionic strength of the composition excluding the contribution of the engineered dimeric protein is less than 10 mM.

In some embodiments of the aqueous solution composition disclosed herein, the pH is between 6.8 and 7.8, for example between 7.0 and 7.8, between 7.1 and 7.6, between 7.1 and 7.5, between 7.2 and 7.5, between 7.1 and 7.4, between 7.2 and 7.3; or is about 7.2 or about 7.3.

In some embodiments, the aqueous solution composition disclosed herein comprises a non-ionic surfactant. In some embodiments, the non-ionic surfactant is selected from the group consisting of an alkyl glycoside, a polysorbate, an alkyl ether of polyethylene glycol, a block copolymer of polyethylene glycol and polypropylene glycol (poloxamer), and an alkylphenyl ether of polyethylene glycol. In some embodiments, the non-ionic surfactant is a polysorbate such as polysorbate 20 or polysorbate 80. In some embodiments, the non-ionic surfactant is a block copolymer of polyethylene glycol and polypropylene glycol (poloxamer), such as poloxamer 188.

In some embodiments of the aqueous solution composition disclosed herein, the non-ionic surfactant is present at a concentration of 10-2000 μg/ml, such as 50-1000 μg/ml, e.g. 100-500 μg/ml e.g. about 200 μg/ml or wherein the non-ionic surfactant is present at a concentration of 250-1500 μg/ml e.g. 750-1250 μg/ml e.g. about 1000 μg/ml. In some embodiments, the aqueous solution composition disclosed herein comprises a preservative such as a phenolic or benzylic preservative. In some embodiments, the phenolic or benzylic preservative is selected from the group consisting of phenol, m-cresol, chlorocresol, benzyl alcohol, propyl paraben and methyl paraben.

In some embodiments of the aqueous solution composition disclosed herein, the preservative is present at a concentration of 10-100 mM, such as 20-80 mM e.g. 25-50 mM.

In some embodiments, the aqueous solution composition disclosed herein is a composition for use in therapy.

In some embodiments, the aqueous solution composition disclosed herein is a pharmaceutical composition.

The present disclosure also provides a method of treating or alleviating a symptom of a disease or disorder associated with aberrant serine protease expression or activity in a subject in need thereof, the method comprising administering an aqueous solution composition disclosed herein.

The present disclosure also provides a method of treating or alleviating inflammation or a symptom of an inflammatory disease or disorder while reducing the risk of infection, in a subject in need thereof, the method comprising administering to said subject an aqueous solution composition disclosed herein.

The present disclosure also provides a method of reducing the risk of infection in a subject in need thereof, the method comprising administering to said subject an aqueous solution composition disclosed herein.

The present disclosure also provides a method of treating or alleviating a symptom of AAT deficiency in a subject in need thereof, the method comprising administering to said subject an aqueous solution composition of the present disclosure, wherein the human serpin polypeptide is a human alpha-1 antitrypsin (AAT) polypeptide or is derived from a human AAT polypeptide.

Also provided herein, is an aqueous solution composition of the present disclosure, for use in a method of treating or alleviating a symptom of a disease or disorder associated with aberrant serine protease expression or activity in a subject in need thereof.

Also provided herein, is an aqueous solution composition of the present disclosure, for use in a method of treating or alleviating inflammation or a symptom of an inflammatory disease or disorder while reducing the risk of infection, in a subject in need thereof.

Also provided herein, is an aqueous solution composition of the present disclosure, for use in a method of reducing the risk of infection in a subject in need thereof.

Also provided herein is an aqueous solution composition of the present disclosure, for use in a method of treating or alleviating a symptom of AAT deficiency in a subject in need thereof, wherein the human serpin polypeptide is a human alpha-1 antitrypsin (AAT) polypeptide or is derived from a human AAT polypeptide.

The present disclosure also provides, use of an aqueous solution composition of the present disclosure, for the manufacture of a medicament for treating or alleviating a symptom of a disease or disorder associated with aberrant serine protease expression or activity in a subject in need thereof.

The present disclosure also provides, use of an aqueous solution composition of the present disclosure, for the manufacture of a medicament for treating or alleviating inflammation or a symptom of an inflammatory disease or disorder while reducing the risk of infection, in a subject in need thereof.

The present disclosure also provides, use of an aqueous solution composition of the present disclosure, for the manufacture of a medicament for reducing the risk of infection in a subject in need thereof.

The present disclosure also provides, use of an aqueous solution composition of the present disclosure, for the manufacture of a medicament for treating or alleviating a symptom of AAT deficiency in a subject in need thereof, wherein the human serpin polypeptide is a human alpha-1 antitrypsin (AAT) polypeptide or is derived from a human AAT polypeptide.

In some embodiments of the aqueous solution composition of the present disclosure for use, or use, according to any of the methods of the present disclosure, the inflammatory disease or disorder is selected from the following: alpha-1 antitrypsin (AAT) deficiency, alpha-1 antitrypsin (AAT) deficiency, emphysema, chronic obstructive pulmonary disease (COPD), acute respiratory distress syndrome (ARDS), allergic asthma, cystic fibrosis, cancers of the lung, ischemia-reperfusion injury, ischemia/reperfusion injury following cardiac transplantation, myocardial infarction, rheumatoid arthritis, septic arthritis, psoriatic arthritis, ankylosing spondylitis, Crohn's disease, psoriasis, type I and/or type II diabetes, pneumonia, sepsis, graft versus host disease (GVHD), wound healing, systemic lupus erythematosus, and multiple sclerosis.

In some embodiments of the aqueous solution composition of the present disclosure for use, or use, according to any of the methods of the present disclosure, the infection is selected from bacterial infections, fungal infections and viral infections.

In some embodiments of the aqueous solution composition of the present disclosure for use, or use, according to any of the methods of the present disclosure, the subject is a human.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### DETAILED DESCRIPTION OF THE INVENTION

Described herein are stable aqueous solution compositions of the engineered dimeric protein of the invention having absent or a low concentration of buffer and low ionic strength.

It should be noted that all references herein to “pH” refer to the pH of a composition evaluated at 25° C. All references to “pKₐ” refer to the pKₐ of an ionisable group evaluated at 25° C. (see CRC Handbook of Chemistry and Physics, 79th Edition, 1998, D. R. Lide). If required, pKₐ values of amino acid side chains as they exist in a polypeptide can be estimated using a suitable calculator.

The present inventors believe that buffers have a detrimental impact on the engineered dimeric protein of the invention. Therefore, the concentration of buffer in the composition should be limited as much as possible.

The buffer(s) where present will have buffering capacity at the pH of the composition. Buffers typically comprise ionisable groups with pKₐ within 1 pH unit of the pH of the composition, however, a moiety which has ionisable groups with pKₐ 1 pH unit greater or less than the pH of the composition may also provide some buffering effect if present in a sufficient amount. In one embodiment, the (or a) buffer comprises ionisable groups with pKₐ within 1 pH unit of the pH of the composition. In another embodiment, the (or a) buffer comprises ionisable groups with pKₐ within 1.5 pH units of the pH of the composition (such as between 1 and 1.5 pH units of the pH of the composition). In a further embodiment, the (or a) buffer comprises ionisable groups with pKₐ within 2 pH units of the pH of the composition (such as between 1.5 and 2 pH units of the pH of the composition).

In an embodiment, the composition is substantially free of buffers. As used herein, “substantially free” means the aqueous solution composition contains less than 0.1 mM of buffers e.g. does not contain any buffers. More preferably, a small amount of buffer is present in order to avoid or limit pH fluctuation which is not desirable. In an embodiment, the composition contains a single buffer. In an embodiment, the composition contains two buffers. Suitably, one or more buffers are present.

The total concentration of buffers in the composition is 0-10 mM. In one embodiment, the total concentration of buffers in the composition is 9.5 mM or less, such as 9 mM or less, such as 8 mM or less, such as 7 mM or less, such as 6 mM or less, such as 5.5 mM or less, such as 5 mM or less, such as 4.5 mM or less, such as 4 mM or less, such as 3 mM or less, such as 2 mM or less, such as 1 mM or less, such as 0.5 mM or less, such as 0.4 mM or less, such as 0.3 mM or less, such as 0.2 mM or less. In one embodiment, the total concentration of buffers is 0.1 mM or more, such as 0.2 mM or more, such as 0.3 mM or more, such as 0.4 mM or more, such as 0.5 mM or more, such as 1 mM or more. Suitably the total concentration of buffers is 0.1-10 mM, such as 0.5-10 mM, such as 1-10 mM, such as 1-8 mM, such as 1-6 mM, such as 2-6 mM, such as 2-5 mM, e.g. 3-5 mM.

When considering the concentration of buffer in solution, any buffering capacity of the engineered dimeric protein of the invention itself should be excluded.

The pH of an aqueous solution decreases if an acid is added and increases if a base is added. At a given temperature and atmospheric pressure, the magnitude of the pH decrease on addition of an acid or the magnitude of the pH increase on addition of a base depends on (1) the amount of the acid or the base added, (2) the starting pH of the aqueous solution (i.e. prior to the addition of the acid or the base) and (3) the presence of a buffer. Thus, (1) starting from a given pH, the addition of a greater amount of an acid or a base will result in greater magnitude of pH change, (2) addition of a given amount of an acid or a base will result in the greatest pH change at neutral pH (i.e. pH 7.0) and the magnitude of the pH change will decrease as the starting pH moves away from pH 7.0 and (3) the magnitude of the pH change, starting from a given pH, will be smaller in the presence of a buffer than in the absence of a buffer. A buffer thus has the ability to reduce the change in pH if an acid or a base is added to the solution.

Suitably, a substance is considered to be a buffer if it is capable of reducing the magnitude of the pH change of a solution to 75%, preferably 50%, most preferably to 25%, compared with an identical solution that does not comprise the buffer, when either strong acid or a strong base is added resulting in 0.1 mM increase of the acid or the base in the solution.

Conversely, suitably, a substance is not considered to be a buffer if it is not capable of reducing the magnitude of the pH change of a solution to 75%, preferably 50%, most preferably to 25%, compared with an identical solution that does not comprise the substance, when either strong acid or a strong base is added resulting in 0.1 mM increase of the acid or the base in the solution.

In one embodiment, the or a buffer is an amino acid. In another embodiment, the or a buffer is not an amino acid. In an embodiment the composition is free of the amino acids lysine, arginine, histidine, glutamate and aspartate. In an embodiment the composition is free of cysteine.

Where present, suitable buffers include, but are not limited to: citrate, histidine, maleate, sulphite, aspartame, aspartate, glutamate, tartrate, adenine, succinate, ascorbate, benzoate, phenylacetate, gallate, cytosine, p-aminobenzoic acid, sorbate, acetate, propionate, alginate, urate, 2-(N-morpholino)ethanesulphonic acid, bicarbonate, bis(2-hydroxyethyl) iminotris(hydroxymethyl)methane, N-(2-acetamido)-2-iminodiacetic acid, 2-[(2-amino-2-oxoethyl)amino]ethanesulphonic acid, piperazine, N,N′-bis(2-ethanesulphonic acid), phosphate, N,N-bis(2-hydroxyethyl)-2-aminoethanesulphonic acid, 3-[N,N-bis(2-hydroxyethyl)amino]-2-hydroxypropanesulphonic acid, triethanolamine, piperazine-N,N′-bis(2-hydroxypropanesulphonic acid), tris(hydroxymethyl)aminomethane (TRIS), N-tris(hydroxymethyl)glycine and N-tris(hydroxymethyl)methyl-3-aminopropanesulphonic acid, and salts thereof, and combinations thereof.

In one embodiment, the buffer is selected from the group consisting of citrate, histidine, maleate, tartrate, benzoate, acetate, bicarbonate, phosphate and TRIS e.g. is selected from the group consisting of histidine, maleate, tartrate, benzoate, acetate, bicarbonate, phosphate and TRIS e.g. is selected from the group consisting of histidine, acetate, phosphate and TRIS.

In one embodiment, the buffer is phosphate (e.g. sodium phosphate) or TRIS. In one embodiment, the buffer is phosphate (e.g. sodium phosphate). Suitably the buffer is TRIS.

In an embodiment, the composition does not comprise sodium phosphate.

The principal solvent for compositions of the invention is water, such as water for injection. Other components of the compositions (e.g. a polyol) may contribute to solubilisation of the engineered dimeric protein.

The composition comprises an uncharged tonicity modifier, such as a polyol, a sugar e.g. a monosaccharide or disaccharide, or a sugar alcohol. In one embodiment, the composition comprises a tonicity modifier selected from the group consisting of glycerol, 1,2-propanediol, mannitol, sorbitol, glucose, sucrose, trehalose, PEG300 and PEG400 e.g. selected from the group consisting of glycerol, 1,2-propanediol, mannitol, sorbitol, sucrose, trehalose, PEG300 and PEG400. Mixtures of uncharged tonicity modifiers such as trehalose and sucrose, or trehalose and mannitol are contemplated. In one embodiment, the uncharged tonicity modifier is selected from glycerol, mannitol, sucrose and trehalose. Suitably the uncharged tonicity modifier is a disaccharide. Thus, in another embodiment, the uncharged tonicity modifier is sucrose and/or trehalose, and in particular is trehalose. When included, an uncharged tonicity modifier (or combination of more than one tonicity modifier) is typically employed in the composition at a total concentration of 50-1000 mM, for example 200-600 mM, such as about 200-500 mM. In one embodiment, the total concentration of uncharged tonicity modifier (or combination of more than one tonicity modifier) is 50-500 mM, such as 100-400 mM, 150-350 mM, 200-300 mM or about 250 mM. Another concentration of interest is about 150 mM.

In an embodiment, when trehalose is the uncharged tonicity modifier, whether alone or in combination with one or more other uncharged tonicity modifiers, the concentration of trehalose in the composition is 50-180 mM e.g., 50-150 mM.

The composition suitably has an osmolarity which is physiologically acceptable and thus suitable for parenteral administration. Thus, the osmolarity of the composition is suitably 200-500 mOsm/L e.g., about 300 mOsm/L. The composition is, for example, isotonic with human plasma. In another embodiment, the osmolarity of the composition is 300-500 mOsm/L e.g. about 400-460 mOsm/L. Compositions may also be hypotonic, or hypertonic, e.g. those intended for dilution prior to administration.

The composition comprises a neutral amino acid. As used herein, a neutral amino acid is an amino acid the side chain of which does not contain an ionisable group which is significantly ionized (e.g. more than 20% especially more than 50% of the side chain have a minus or plus charge) at the pH of the composition. Example neutral amino acids are glycine, methionine, proline, alanine, valine, leucine, isoleucine, phenylalanine, tyrosine, tryptophan, serine, threonine, asparagine and glutamine and in particular the L isomers thereof.

In one embodiment, the composition comprises a neutral amino acid selected from the group consisting of glycine, methionine and proline. In one embodiment, the composition comprises proline as neutral amino acid. In one embodiment, the composition comprises glycine as neutral amino acid. Mixtures of neutral amino acids are contemplated. In one embodiment, the composition comprises proline and methionine as neutral amino acids. In one embodiment, the composition comprises glycine and methionine as neutral amino acids. As can be seen from the examples, the presence of a neutral amino acid was found to enhance the stability of the composition.

In one embodiment, the concentration of the neutral amino acid, for example, proline or glycine, is 20 to 600 mM, such as 20 to 500 mM, such as 20 to 400 mM, such as 20 to 300 mM or 50 to 300 mM. In one embodiment, the concentration of the neutral amino acid, for example, proline or glycine, is 50 to 200 mM, 100 to 200 mM or 100 to 150 mM.

In one embodiment, methionine as a neutral amino acid, when present in the composition, is present at a concentration of 2 to 10 mM, e.g. about 2 mM.

In one embodiment, the concentration of all the neutral amino acids (i.e. the total concentration) in the composition, for example, proline or glycine and methionine, is 20 to 600 mM, such as 20 to 500 mM, such as 20 to 400 mM, such as 20 to 300 mM or 50 to 300 mM. In one embodiment, the concentration of all the neutral amino acids in the composition, for example, proline or glycine and methionine, is 50 to 200 mM, 100 to 200 mM or 100 to 150 mM.

The composition may comprise a non-ionic surfactant. The non-ionic surfactant may for example be selected from the group consisting of a polysorbate, an alkyl glycoside, an alkyl ether of polyethylene glycol, a block copolymer of polyethylene glycol and polypropylene glycol, and an alkylphenyl ether of polyethylene glycol.

A particularly suitable class of non-ionic surfactants is the polysorbates (fatty acid esters of ethoxylated sorbitan), such as polysorbate 20 or polysorbate 80. Polysorbate 20 is a mono ester formed from lauric acid and polyoxyethylene (20) sorbitan in which the number 20 indicates the number of oxyethylene groups in the molecule. Polysorbate 80 is a mono ester formed from oleic acid and polyoxyethylene (20) sorbitan in which the number 20 indicates the number of oxyethylene groups in the molecule. Polysorbate 20 is known under a range of brand names including in particular Tween 20, and also Alkest TW 20. Polysorbate 80 is known under a range of brand names including in particular Tween 80, and also Alkest TW 80. Other suitable polysorbates include polysorbate 40 and polysorbate 60.

Another suitable class of non-ionic surfactants is the alkyl glycosides, especially dodecyl maltoside. Other alkyl glycosides include dodecyl glucoside, octyl glucoside, octyl maltoside, decyl glucoside, decyl maltoside, tridecyl glucoside, tridecyl maltoside, tetradecyl glucoside, tetradecyl maltoside, hexadecyl glucoside, hexadecyl maltoside, sucrose monooctanoate, sucrose mono decanoate, sucrose monododecanoate, sucrose monotridecanoate, sucrose monotetradecanoate and sucrose monohexadecanoate.

Another suitable class of non-ionic surfactants is alkyl ethers of polyethylene glycol, especially those known under a brand name Brij, such as selected from polyethylene glycol (2) hexadecyl ether (Brij 52), polyethylene glycol (2) oleyl ether (Brij 93) and polyethylene glycol (2) dodecyl ether (Brij L4). Other suitable Brij surfactants include polyethylene glycol (4) lauryl ether (Brij 30), polyethylene glycol (10) lauryl ether (Brij 35), polyethylene glycol (20) hexadecyl ether (Brij 58) and polyethylene glycol (10) stearyl ether (Brij 78).

Another suitable class of non-ionic surfactants is block copolymers of polyethylene glycol and polypropylene glycol, also known as poloxamers, especially poloxamer 188, poloxamer 407, poloxamer 171 and poloxamer 185. Poloxamers are also known under brand names Pluronics or Koliphors. For example, poloxamer 188 is marketed as Pluronic F-68.

Another suitable class of non-ionic surfactants are alkylphenyl ethers of polyethylene glycol, especially 4-(1,1,3,3-tetramethylbutyl)phenyl-polyethylene glycol, also known under a brand name Triton X-100.

In one embodiment, the non-ionic surfactant is a polysorbate or a poloxamer. In one embodiment, the non-ionic surfactant is a polysorbate, such as polysorbate 80 or polysorbate 20. In one embodiment, the non-ionic surfactant is a poloxamer, such as poloxamer 188. The concentration of the non-ionic surfactant in the composition will typically be in the range 10-2000 μg/ml. Exemplary concentrations e.g. for polysorbate surfactants are 50-1000 μg/ml, e.g. 100-500 μg/ml e.g. about 200 μg/ml. Exemplary concentrations e.g. for poloxamer surfactants are 250-1500 μg/ml, e.g. 750-1250 μg/ml e.g. about 1000 μg/ml.

In some embodiments, the poloxamer surfactants concentration are 0.025%-0.15% by weight per volume (w/v) of the composition, e.g. 0.075% to 0.125% by weight per volume (w/v) of the composition, e.g. about 0.1% by weight per volume (w/v) of the composition.

The compositions of the invention may additionally comprise a preservative such as a phenolic or a benzylic preservative. The preservative is suitably selected from the group consisting of phenol, m-cresol, chlorocresol, benzyl alcohol, propyl paraben and methyl paraben, in particular phenol, m-cresol and benzyl alcohol. The concentration of preservative is typically 10-100 mM, for example 20-80 mM, such as 25-50 mM. The optimal concentration of the preservative in the composition is selected to ensure the composition passes the Pharmacopoeia Antimicrobial Effectiveness Test (USP <51>, Vol. 32).

The present inventors believe that the presence of ions has a detrimental impact on the stability of the engineered dimeric protein of the invention. Therefore, the ionic strength of the composition should be limited as much as possible.

The total ionic strength of the composition excluding the contribution of the engineered dimeric protein of the invention is less than 30 mM, suitably less than 25 mM, suitably less than 20 mM, suitably less than 15 mM, suitably less than 10 mM e.g. less than 5 mM. The term “total ionic strength” is used herein as the following function of the concentration of all ions in a solution:

$$I = {\sum }_{X = 1}^{n} {c}_{x} {z}_{x}^{2} / 2$$

where cₓ is molar concentration of ion x (mol L⁻¹), zₓ is the net charge of ion cₓ. The sum covers all ions (n) present in the solution excluding the contribution of the engineered dimeric protein of the invention. It will be understood that optional neutral amino acids have a net charge of zero in the compositions of the invention and do not thus contribute to the total ionic strength. In any event, the contribution of any neutral amino acids is not included.

The pH of the composition is between 6.0 and 8.0, such as between 6.8 and 7.8, for example between 7.0 and 7.8, between 7.1 and 7.6, between 7.1 and 7.5, between 7.2 and 7.5, between 7.1 and 7.4, between 7.2 and 7.3; or is about 7.2 or about 7.3.

In certain embodiments, the engineered dimeric protein of the invention is substantially pure, that is, the composition comprises a single protein and no substantial amount of any additional protein. In preferred embodiments, the engineered dimeric protein of the invention comprises at least 99%, preferably at least 99.5% and more preferably at least about 99.9% of the total protein content of the composition. In preferred embodiments the engineered dimeric protein of the invention is sufficiently pure for use in a pharmaceutical composition.

The engineered dimeric protein of the invention is suitably present in the composition at a concentration of about 1-400 mg/ml, suitably 10-200 mg/ml, more suitably 20-100 mg/ml e.g. 30-60 mg/ml e.g. about 35 mg/ml or about 50 mg/ml.

In one embodiment, the invention provides an aqueous solution composition of pH in the range 6.8 to 7.8 e.g. 7.0 to 7.8 e.g. 7.1 to 7.6 e.g. 7.1 to 7.5 e.g. about 7.2 or about 7.3 comprising: an engineered dimeric protein wherein each monomer of the dimeric protein comprises at least one human serpin polypeptide operably linked to a human immunoglobulin Fc polypeptide or a polypeptide that is derived from an immunoglobulin Fc polypeptide; a buffer selected from phosphate (e.g. sodium phosphate) or TRIS, e.g. TRIS; a neutral amino acid selected from the group consisting of glycine, methionine and proline e.g. glycine and methionine or proline and methionine; and an uncharged tonicity modifier e.g. a disaccharide e.g. trehalose, sucrose or a mixture of trehalose and sucrose; a non-ionic surfactant selected from a polysorbate and a poloxamer e.g. a poloxamer such as poloxamer 188; wherein buffers are present in the composition at a total concentration of 1-8 mM e.g. 2-6 mM; and wherein the total ionic strength of the composition excluding the contribution of the engineered dimeric protein is less than 20 mM e.g. less than 10 mM e.g. less than 5 mM.

Suitably, the composition of the invention remains as a clear solution following storage at 2-8° C. for extended period of time, such as at least 4 weeks, 8 weeks, 12 weeks, 12 months, 18 months or 24 months.

Suitably, the composition of the invention remains as a clear solution following storage at 25° C. for extended period of time, such as at least 4 weeks, 8 weeks, 12 weeks, 12 months, 18 months or 24 months.

Suitably, the composition of the invention remains as a clear solution following storage at 30° C. for extended period of time, such as at least 4 weeks, 8 weeks, 12 weeks, 12 months, 18 months or 24 months.

Suitably, the composition of the invention remains as a clear solution following storage at 40° C. (i.e. temperature suitable for accelerated stability trials) for a period of time, such as at least 1 day, 3 days, 1 week, 2 weeks or 4 weeks.

Suitably, the composition of the invention has increased storage stability either at 2-8° C. or at increased temperature as compared to an equivalent composition that comprises higher concentration of the same buffer or buffers.

Suitably, the composition of the invention has increased storage stability either at 2-8° C. or at increased temperature as compared to an equivalent composition that has a higher total ionic strength.

In one embodiment, the composition of the invention comprises no more than 8% high molecular weight species, such as no more than 7%, such as no more than 6%, such as no more than 5%, such as no more than 2%, such as no more than 1%, such as no more than 0.5%, such as no more than 0.3% high molecular weight species (by total weight of the engineered dimeric protein of the invention in the composition, as measured by size-exclusion chromatography or a similar suitable technique) following storage at 2-8° C. for at least 4 weeks, 8 weeks, 12 weeks, 12 months, 18 months or 24 months.

In one embodiment, the composition of the invention comprises no more than 18% high molecular weight species, such as no more than 17%, such as no more than 16%, such as no more than 15%, such as no more than 10%, such as no more than 8%, such as no more than 5%, such as no more than 4%, such as no more than 3%, such as no more than 1% high molecular weight species (by total weight of the engineered dimeric protein of the invention in the composition, as measured by size-exclusion chromatography or a similar suitable technique) following storage at 25° C. for at least 4 weeks, 8 weeks, 12 weeks, 12 months, 18 months or 24 months.

In one embodiment, the composition of the invention comprises no more than 25% high molecular weight species, such as no more than 20%, such as no more than 18%, such as no more than 17%, such as no more than 16%, such as no more than 15%, such as no more than 10%, such as no more than 8%, such as no more than 5%, such as no more than 4%, such as no more than 3%, such as no more than 1% high molecular weight species (by total weight of the engineered dimeric protein of the invention in the composition, as measured by size-exclusion chromatography or a similar suitable technique) following storage at 30° C. for at least 4 weeks.

In one embodiment, the composition of the invention comprises no more than 30% high molecular weight species, such as no more than 25%, such as no more than 20%, such as no more than 18%, such as no more than 17%, such as no more than 16%, such as no more than 15%, no more than 10%, such as no more than 8%, such as no more than 6%, such as no more than 4% high molecular weight species (by total weight of the engineered dimeric protein of the invention in the composition, as measured by size-exclusion chromatography or a similar suitable technique) following storage at 40° C. for at least 1 day, 3 days, 1 week, 2 weeks or 4 weeks.

As used herein, high molecular weight species are species that result from protein aggregation with an apparent molecular weight greater than the dimeric protein.

In an embodiment, the composition of the invention is a composition for use in therapy. In an embodiment, the composition of the invention is a pharmaceutical composition.

In one embodiment is provided a method of inhibiting or downregulating aberrant serine protease expression or activity in a subject in need thereof, the method comprising administering to said subject an aqueous solution composition as described herein. In some embodiments, the aberrant serine protease expression or activity is associated with an inflammatory disease or disorder or a risk of infection. In some embodiments, the inflammatory disease or disorder is selected from the following: alpha-1 antitrypsin (AAT) deficiency, emphysema, chronic obstructive pulmonary disease (COPD), acute respiratory distress syndrome (ARDS), allergic asthma, cystic fibrosis, cancers of the lung, ischemia-reperfusion injury, ischemia/reperfusion injury following cardiac transplantation, myocardial infarction, rheumatoid arthritis, septic arthritis, psoriatic arthritis, ankylosing spondylitis, Crohn's disease, psoriasis, type I and/or type II diabetes, pneumonia, sepsis, graft versus host disease (GVHD), wound healing, systemic lupus erythematosus, and multiple sclerosis.

In one embodiment, the risk of infection is risk of an infection selected from bacterial infections, fungal infections and viral infections.

In one embodiment is provided a method of treating or alleviating a symptom of a disease or disorder associated with aberrant serine protease expression or activity in a subject in need thereof, the method comprising administering to said subject an aqueous solution composition as described herein. In another embodiment is provided a method of treating or alleviating inflammation or a symptom of an inflammatory disease or disorder while reducing the risk of infection, in a subject in need thereof, the method comprising administering to said subject an aqueous solution composition as described herein. In another embodiment is provided a method of reducing the risk of infection in a subject in need thereof, the method comprising administering to said subject an aqueous solution composition as described herein.

In an embodiment, when the human serpin polypeptide is a human alpha-1 antitrypsin (AAT) polypeptide or is derived from a human AAT polypeptide, there is provided a method of treating or alleviating a symptom of AAT deficiency in a subject in need thereof, the method comprising administering to said subject an aqueous solution composition as described herein.

In one embodiment is provided an aqueous solution composition as described herein, for use in a method of treating or alleviating a symptom of a disease or disorder associated with aberrant serine protease expression or activity in a subject in need thereof. In another embodiment is provided an aqueous solution composition as described herein, for use in a method of treating or alleviating inflammation or a symptom of an inflammatory disease or disorder while reducing the risk of infection, in a subject in need thereof. In another embodiment is provided an aqueous solution composition as described herein, for use in a method of reducing the risk of infection in a subject in need thereof.

In an embodiment, when the human serpin polypeptide is a human alpha-1 antitrypsin (AAT) polypeptide or is derived from a human AAT polypeptide, there is provided an aqueous solution composition as described herein for use in a method of treating or alleviating a symptom of AAT deficiency in a subject in need thereof.

In one embodiment is provided the use of an aqueous solution composition as described herein, for the manufacture of a medicament for treating or alleviating a symptom of a disease or disorder associated with aberrant serine protease expression or activity in a subject in need thereof. In another embodiment is provided the use of an aqueous solution composition as described herein, for the manufacture of a medicament for treating or alleviating inflammation or a symptom of an inflammatory disease or disorder while reducing the risk of infection, in a subject in need thereof. In another embodiment is provided the use of an aqueous solution composition as described herein, for the manufacture of a medicament for reducing the risk of infection in a subject in need thereof.

In an embodiment, when the human serpin polypeptide is a human alpha-1 antitrypsin (AAT) polypeptide or is derived from a human AAT polypeptide, there is provided use of an aqueous solution composition as described herein for the manufacture of a medicament for treating or alleviating a symptom of AAT deficiency in a subject in need thereof.

Suitably, the subject is a human.

Suitably, the inflammatory disease or disorder is selected from the following: alpha-1 antitrypsin (AAT) deficiency, emphysema, chronic obstructive pulmonary disease (COPD), acute respiratory distress syndrome (ARDS), allergic asthma, cystic fibrosis, cancers of the lung, ischemia-reperfusion injury, ischemia/reperfusion injury following cardiac transplantation, myocardial infarction, rheumatoid arthritis, septic arthritis, psoriatic arthritis, ankylosing spondylitis, Crohn's disease, psoriasis, type I and/or type II diabetes, pneumonia, sepsis, graft versus host disease (GVHD), wound healing, systemic lupus erythematosus, and multiple sclerosis.

Suitably, the infection is selected from bacterial infections, fungal infections and viral infections.

Compositions e.g. those intended for intravenous administration may be prepared as concentrates for dilution prior to administration.

All embodiments described above with respect to the aqueous solution composition apply equally to methods and uses of the invention.

There is also provided a container, for example made of plastics or glass, containing one dose or a plurality of doses of the composition as described herein. The container can be for example, a vial, a pre-filled syringe, a pre-filled infusion bag, or a cartridge designed to be a replaceable item for use with an injection device.

The compositions of the invention may suitably be packaged for infusion or injection, especially intravenous infusion, intravenous injection, subcutaneous injection or intramuscular injection.

The compositions of the invention may suitably be packed in a vial as a concentrate for intravenous infusion. Prior to use, the concentrate is removed from the vial and diluted into an infusion bag containing a suitable diluent such as saline solution, dextrose solution or water for injection. The diluted composition is subsequently administered by intravenous infusion at a specified infusion rate (e.g., 8-16 mL/min).

An aspect of the invention is an injection or infusion device, particularly a device adapted for subcutaneous or intramuscular injection or infusion, for single or multiple use comprising a container containing one dose or a plurality of doses of the composition of the invention together with an injection needle. In an embodiment, the container is a replaceable cartridge which contains a plurality of doses. In one embodiment, the injection device is in the form of a pen. In one embodiment, the injection device is in the form of a pre-filled syringe. In one embodiment, the injection or infusion device is in the form of a pump or another wearable injection or infusion device.

Compositions according to the invention are expected to have good physical and chemical stability as described herein.

### DESCRIPTION OF THE SEQUENCE LISTING

SEQ ID NO: 1 is a full-length human AAT polypeptide sequence.

SEQ ID NO: 2 is a full-length human AAT polypeptide sequence.

SEQ ID NO: 3 is a reactive site loop portion of the AAT protein.

SEQ ID NO: 4 is a reactive site loop portion of the AAT protein.

SEQ ID NO: 5 is a reactive site loop portion of the AAT protein.

SEQ ID NO: 6 is a human IgG1 Fc polypeptide sequence.

SEQ ID NO: 7 is a human IgG1 Fc polypeptide sequence including a hinge region at the N-terminus.

SEQ ID NO: 8 is a modified IgG1 Fc polypeptide sequence with mutations at residues M252, T256 and M428.

SEQ ID NO: 9 is a modified IgG1 Fc polypeptide sequence including a hinge region at the N-terminus, with mutations at residues M252, T256 and M428.

SEQ ID NO: 10 is a modified IgG1 Fc polypeptide sequence where residue G236 is deleted.

SEQ ID NO: 11 is a modified IgG1 Fc polypeptide sequence including a hinge region at the N-terminus, where residue G236 is deleted.

SEQ ID NO: 12 is a modified IgG1 Fc polypeptide sequence with mutations at residues L234 and L235.

SEQ ID NO: 13 is a modified IgG1 Fc polypeptide sequence including a hinge region at the N-terminus, with mutations at residues L234 and L235.

SEQ ID NO: 14 is a modified IgG1 Fc polypeptide sequence with a deletion at residue G236 and mutations at residues L234 and L235.

SEQ ID NO: 15 is a modified IgG1 Fc polypeptide sequence including a hinge region at the N-terminus, with a deletion at residue G236 and mutations at residues L234 and L235.

SEQ ID NO: 16 is a modified IgG1 Fc polypeptide sequence with a deletion at residue G236 and mutations at residues L234, L235, M252, T256, and M428.

SEQ ID NO: 17 is a modified IgG1 Fc polypeptide sequence including a hinge region at the N-terminus, with a deletion at residue G236 and mutations at residues L234, L235, M252, T256, and M428.

SEQ ID NO: 18 is a human IgG2 Fc polypeptide sequence.

SEQ ID NO: 19 is a modified IgG2 Fc polypeptide sequence with a deletion at residue G236 and mutations at residues M252, T256, and M428.

SEQ ID NO:20 is a human IgG3 Fc polypeptide sequence.

SEQ ID NO: 21 is a modified IgG3 Fc polypeptide sequence with mutations at residues M252, T256, and M428.

SEQ ID NO: 22 is a modified IgG3 Fc polypeptide sequence with a deletion at residue G236.

SEQ ID NO: 23 is a modified IgG3 Fc polypeptide sequence with mutations at residues L234 and L235.

SEQ ID NO: 24 is a modified IgG3 Fc polypeptide sequence with a deletion at residue G236 and mutations at residues L234 and L235.

SEQ ID NO: 25 is a modified IgG3 Fc polypeptide sequence with a deletion at residue G236 and mutations at residues L234, L235, M252, T256, and M428.

SEQ ID NO: 26 is a human IgG4 Fc polypeptide sequence.

SEQ ID NO: 27 is a human IgG4 Fc polypeptide sequence including a hinge region at the N-terminus.

SEQ ID NO: 28 is a modified IgG4 Fc polypeptide sequence with mutations at residues M252, T256 and M428.

SEQ ID NO: 29 is a modified IgG4 Fc polypeptide sequence including a hinge region at the N-terminus, with mutations at residues M252, T256 and M428.

SEQ ID NO: 30 is a modified IgG4 Fc polypeptide sequence with a deletion at residue G236.

SEQ ID NO: 31 is a modified IgG4 Fc polypeptide sequence including a hinge region at the N-terminus, with a deletion at residue G236.

SEQ ID NO: 32 is a modified IgG4 Fc polypeptide sequence with a mutation at residue L235.

SEQ ID NO: 33 is a modified IgG4 Fc polypeptide sequence including a hinge region at the N-terminus, with a mutation at residue L235.

SEQ ID NO: 34 is a modified IgG4 Fc polypeptide sequence with mutations at residues L234 and L235.

SEQ ID NO: 35 is a modified IgG4 Fc polypeptide sequence including a hinge region at the N-terminus, with mutations at residues L234 and L235.

SEQ ID NO: 36 is a modified IgG4 Fc polypeptide sequence with a mutation at residue S228.

SEQ ID NO: 37 is a modified IgG4 Fc polypeptide sequence including a hinge region at the N-terminus, with mutations at residues S228 and L235.

SEQ ID NO: 38 is a modified IgG4 Fc polypeptide sequence including a hinge region at the N-terminus, with mutations at residues S228, L235 and M252.

SEQ ID NO: 39 is a modified IgG4 Fc polypeptide sequence including a hinge region at the N-terminus, with mutations at residues S228, L235 and M428.

SEQ ID NO: 40 is a modified IgG4 Fc polypeptide sequence including a hinge region at the N-terminus, with mutations at residues S228, L235, M252 and M428.

SEQ ID NO: 41 is a modified IgG4 Fc polypeptide sequence with mutations at residues L235, M252, T256 and M428.

SEQ ID NO: 42 is a modified IgG4 Fc polypeptide sequence including a hinge region at the N-terminus, with mutations at residues L235, M252, T256 and M428.

SEQ ID NO: 43 is a modified IgG4 Fc polypeptide sequence including a hinge region at the N-terminus, with mutations at residues S228, L235, M252, T256 and M428.

SEQ ID NO: 44 is a human IgM Fc polypeptide.

SEQ ID NO: 45 is an AAT polypeptide-IgG-Fc polypeptide fusion protein comprising SEQ ID NO: 1 and SEQ ID NO: 6.

SEQ ID NO: 46 is an AAT polypeptide-IgG-Fc polypeptide fusion protein comprising SEQ ID NO: 1 and SEQ ID NO: 18.

SEQ ID NO: 47 is an AAT polypeptide-IgG-Fc polypeptide fusion protein comprising SEQ ID NO: 48 and SEQ ID NO: 6.

SEQ ID NO: 48 is an AAT polypeptide.

SEQ ID NO: 49 is an AAT polypeptide-IgG-Fc polypeptide fusion protein comprising SEQ ID NO: 48 and SEQ ID NO: 18.

SEQ ID NO: 50 is an AAT polypeptide-IgG-Fc polypeptide fusion protein comprising SEQ ID NO: 51 and SEQ ID NO: 18

SEQ ID NO: 51 is an AAT polypeptide.

SEQ ID NO: 52 is an AAT polypeptide-IgG-Fc polypeptide fusion protein comprising SEQ ID NO: 1 and SEQ ID NO: 6.

SEQ ID NO: 53 is an AAT polypeptide-IgG-Fc polypeptide fusion protein comprising SEQ ID NO: 2 and SEQ ID NO: 17.

SEQ ID NO: 54 is an AAT polypeptide-IgG-Fc polypeptide fusion protein comprising SEQ ID NO: 2 and SEQ ID NO: 43.

SEQ ID NO: 55 is an AAT polypeptide-IgG-Fc polypeptide fusion protein comprising SEQ ID NO: 51 and SEQ ID NO: 7.

SEQ ID NO: 56 is an AAT polypeptide-IgG4-Fc polypeptide fusion protein comprising SEQ ID NO: 48 and SEQ ID NO: 40.

Each monomer of the engineered dimeric protein of the invention comprises at least one human serpin polypeptide (e.g. one or two) operably linked to a human immunoglobulin Fc polypeptide or a polypeptide that is derived from an immunoglobulin Fc polypeptide.

As used herein, “operably linked” means linked as part of a continuous polypeptide chain i.e. as a fusion protein. Such engineered proteins are capable of being prepared by recombinant engineering techniques. As used herein, “operably linked” also means that the at least one human serpin polypeptide in fusion with the immunoglobulin Fc polypeptide is functional in terms of inhibiting serine protease activity.

In an embodiment, the human serpin polypeptide is a human alpha-1 antitrypsin (AAT) polypeptide or is derived from a human AAT polypeptide.

In an embodiment, each monomer of the dimeric protein comprises one human serpin polypeptide.

In an embodiment, each monomer of the engineered dimeric protein comprises at least one (e.g. one) polypeptide selected from a human alpha-1 antitrypsin (AAT) polypeptide and polypeptides which are derived from a human AAT polypeptide wherein said at least one polypeptide is (are) operably linked to the N-terminal end of the human immunoglobulin Fc polypeptide or a polypeptide that is derived from an immunoglobulin Fc polypeptide.

For example, the or each polypeptide which is a human alpha-1 antitrypsin (AAT) polypeptide or is derived from a human AAT polypeptide that has the sequence of SEQ ID NOs: 1 or 2.

For example, the or each polypeptide which is a human alpha-1 antitrypsin (AAT) or is derived from a human AAT polypeptide has the sequence of SEQ ID NO: 1. For example, the or each polypeptide which is a human alpha-1 antitrypsin (AAT) or is derived from a human AAT polypeptide has a sequence that is at least 50%, 60%, 65%, 70%, 75%, 80%, 85%, 90%, 91%, 92%, 93%, 94%, 95%, 96%, 97%, 98% or 99% identical to the amino acid sequence of

SEQ ID NO: 1.

In some embodiments, a full-length human AAT polypeptide sequence has the following amino acid sequence:

(SEQ ID NO: 1)

| | |
|---|---|
| 1 | EDPQGDAAQK TDTSHHDQDH PTFNKITPNL AEFAFSLYRQ |
| | LAHQSNSTNI FFSPVSIATA |
| 61 | FAMLSLGTKA DTHDEILEGL NFNLTEIPEA QIHEGFQELL |
| | RTLNQPDSQL QLTTGNGLFL |
| 121 | SEGLKLVDKF LEDVKKLYHS EAFTVNFGDT EEAKKQINDY |
| | VEKGTQGKIV DLVKELDRDT |
| 181 | VFALVNYIFF KGKWERPFEV KDTEEEDFHV DQVTTVKVPM |
| | MKRLGMFNIQ HCKKLSSWVL |
| 241 | LMKYLGNATA IFFLPDEGKL QHLENELTHD IITKFLENED |
| | RRSASLHLPK LSITGTYDLK |
| 301 | SVLGQLGITK VFSNGADLSG VTEEAPLKLS KAVHKAVLTI |
| | DEKGTEAAGA MFLEAIPMSI |
| 361 | PPEVKFNKPF VFLMIEQNTK SPLFMGKVVN PTQK |

For example, the or each polypeptide which is a human alpha-1 antitrypsin (AAT) or is derived from a human AAT polypeptide has the sequence of SEQ ID NO: 2. For example, the or each polypeptide which is a human alpha-1 antitrypsin (AAT) or is derived from a human AAT polypeptide has a sequence that is at least 50%, 60%, 65%, 70%, 75%, 80%, 85%, 90%, 91%, 92%, 93%, 94%, 95%, 96%, 97%, 98% or 99% identical to the amino acid sequence of SEQ ID NO: 2.

In some embodiments, a full-length human AAT polypeptide sequence has the following amino acid sequence:

| | (SEQ ID NO: 2) |
|---|---|
| 1 | EDPQGDAAQK TDTSHHDQDH PTFNKITPNL AEFAFSLYRQ LAHQSNSTNI FFSPVSIATA |
| 61 | FAMLSLGTKA DTHDEILEGL NFNLTEIPEA QIHEGFQELL RTLNQPDSQL QLTTGNGLFL |
| 121 | SEGLKLVDKF LEDVKKLYHS EAFTVNFGDT EEAKKQINDY VEKGTQGKIV DLVKELDRDT |
| 181 | VFALVNYIFF KGKWERPFEV KDTEEEDFHV DQVTTVKVPM MKRLGMFNIQ HCKKLSSWVL |
| 241 | LMKYLGNATA IFFLPDEGKL QHLENELTHD IITKFLENED RRSASLHLPK LSITGTYDLK |
| 301 | 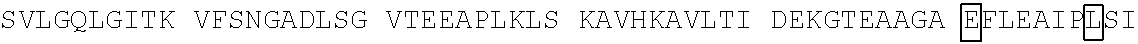 |
| 361 | PPEVKFNKPF VFLMIEQNTK SPLFMGKVVN PTQK |

For example, the or each polypeptide which is a human alpha-1 antitrypsin (AAT) or is derived from a human AAT polypeptide sequences has a sequence shown in GenBank Accession Nos. AAB59495.1, CAJ15161.1, P01009.3, AAB59375.1, AAA51546.1, CAA25838.1, NP_001002235.1, CAA34982.1, NP_001002236.1, NP_000286.3, NP_001121179.1, NP_001121178.1, NP_001121177.1, NP_001121176.16, NP_001121175.1, NP_001121174.1, NP_001121172.1, and/or AAA51547.1.

For example, the or each polypeptide which is a human alpha-1 antitrypsin (AAT) polypeptide comprises a reactive loop sequence according to any one of the sequence of SEQ ID NOs: 3, 4 and 5.

In some embodiments, the reactive site loop portion of the AAT protein includes at least the amino acid sequence: GTEAAGAMFLEAIPMSIPPEVKFNK (SEQ ID NO: 3). In some embodiments, the reactive site loop portion of the AAT protein includes at least the amino acid sequence: GTEAAGAEFLEAIPLSIPPEVKFNK (SEQ ID NO: 4). In some embodiments, the reactive site loop portion of the AAT protein includes at least the amino acid sequence: GTEAAGALFLEAIPLSIPPEVKFNK (SEQ ID NO: 5).

In an embodiment, the polypeptide comprises human immunoglobulin Fc polypeptide or a polypeptide that is derived from an immunoglobulin Fc polypeptide is a human IgG1 Fc polypeptide. In some embodiments the human IgG1 Fc polypeptide sequence has the following amino acid sequence:

(SEQ ID NO: 6)

| | |
|---|---|
| 1 | APELLGGPSV FLFPPKPKDT LMISRTPEVT CVVVDVSHED |
| | PEVKFNWYVD GVEVHNAKTK |
| 61 | PREEQYNSTY RVVSVLTVLH QDWLNGKEYK CKVSNKALPA |
| | PIEKTISKAK GQPREPQVYT |
| 121 | LPPSRDELTK NQVSLTCLVK GFYPSDIAVE WESNGQPENN |
| | YKTTPPVLDS DGSFFLYSKL |
| 181 | TVDKSRWQQG NVFSCSVMHE ALHNHYTQKS LSLSPGK |

In some embodiments, the polypeptide includes a hinge region coupled to the N-terminus of the Fc polypeptide of the polypeptide, where the Fc polypeptide includes a human IgG1 Fc polypeptide sequence having the following amino acid sequence:

(SEQ ID NO: 7)

| | |
|---|---|
| 1 | DKTHTCPPCP APELLGGPSV FLFPPKPKDT LMISRTPEVT |
| | CVVVDVSHED PEVKFNWYVD |
| 61 | GVEVHNAKTK PREEQYNSTY RVVSVLTVLH QDWLNGKEYK |
| | CKVSNKALPA PIEKTISKAK |
| 121 | GQPREPQVYT LPPSRDELTK NQVSLTCLVK GFYPSDIAVE |
| | WESNGQPENN YKTTPPVLDS |
| 181 | DGSFFLYSKL TVDKSRWQQG NVFSCSVMHE ALHNHYTQKS |
| | LSLSPGK |

In some embodiments where the polypeptide of the invention includes an Fc polypeptide, the Fc polypeptide of the polypeptide includes a human IgG1 Fc polypeptide sequence that is at least 50%, 60%, 65%, 70%, 75%, 80%, 85%, 90%, 91%, 92%, 93%, 94%, 95%, 96%, 97%, 98% or 99% identical to the amino acid sequence of SEQ ID NO: 6 or 7.

In some embodiments, the polypeptide of the invention includes a modified IgG1 Fc polypeptide, the modified IgG1 Fc polypeptide of the fusion protein includes mutations at residues M252, T256, and M428, which correspond to residues 22, 26, and 198 of SEQ ID NO: 6 or residues 32, 36, and 208 of SEQ ID NO: 7 shown above, and has the following amino acid sequence, where the mutated amino acid residues are boxed:

| | (SEQ ID NO: 8) |
|---|---|
| 1 | 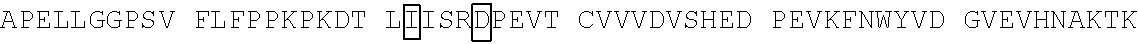 |
| 61 | PREEQYNSTY RVVSVLTVLH QDWLNGKEYK CKVSNKALPA PIEKTISKAK GQPREPQVYT |
| 121 | LPPSRDELTK NQVSLTCLVK GFYPSDIAVE WESNGQPENN YKTTPPVLDS DGSFFLYSKL |
| 181 | 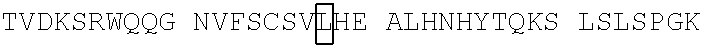 |

In some embodiments, the polypeptide of the invention includes a hinge region coupled to the N-terminus of a modified IgG1 Fc polypeptide, the modified IgG1 Fc polypeptide of the fusion protein includes mutations at residues M252, T256, and M428, which correspond to residues 22, 26, and 198 of SEQ ID NO: 6 or residues 32, 36, and 208 of SEQ ID NO: 7 shown above, and the fusion protein includes at least the following amino acid sequence, where the mutated amino acid residues are boxed:

| | (SEQ ID NO: 9) |
|---|---|
| 1 | 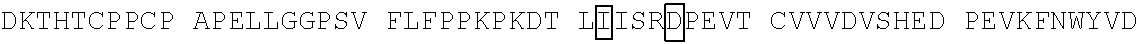 |
| 61 | GVEVHNAKTK PREEQYNSTY RVVSVLTVLH QDWLNGKEYK CKVSNKALPA PIEKTISKAK |
| 121 | GQPREPQVYT LPPSRDELTK NQVSLTCLVK GFYPSDIAVE WESNGQPENN YKTTPPVLDS |
| 181 | 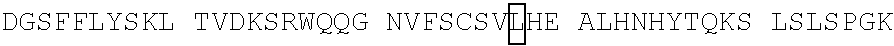 |

In some embodiments, the polypeptide of the invention includes a modified IgG1 Fc polypeptide, the modified IgG1 Fc polypeptide of the fusion protein includes a modified human IgG1 Fc polypeptide sequence where residue G236, which corresponds to residue 6 of SEQ ID NO: 6 or residue 16 of SEQ ID NO: 7 shown above, is deleted and has the following amino acid sequence:

| (SEQ ID NO: 10) | |
|---|---|
| 1 | APELLGPSVF LFPPKPKDTL MISRTPEVTC VVVDVSHEDP EVKFNWYVDG VEVHNAKTKP |
| 61 | REEQYNSTYR VVSVLTVLHQ DWLNGKEYKC KVSNKALPAP IEKTISKAKG QPREPQVYTL |
| 121 | PPSRDELTKN QVSLTCLVKG FYPSDIAVEW ESNGQPENNY KTTPPVLDSD GSFFLYSKLT |
| 181 | VDKSRWQQGN VFSCSVMHEA LHNHYTQKSL SLSPGK |

In some embodiments, the polypeptide of the invention includes a hinge region coupled to the N-terminus of a modified IgG1 Fc polypeptide, the modified IgG1 Fc polypeptide of the fusion protein includes a modified human IgG1 Fc polypeptide sequence where residue G236, which corresponds to residue 6 of SEQ ID NO: 6 or residue 16 of SEQ ID NO: 7 shown above, is deleted, and the fusion protein includes at least the following amino acid sequence:

| (SEQ ID NO: 11) | |
|---|---|
| 1 | DKTHTCPPCP APELLGPSVF LFPPKPKDTL MISRTPEVTC VVVDVSHEDP EVKFNWYVDG |
| 61 | VEVHNAKTKP REEQYNSTYR VVSVLTVLHQ DWLNGKEYKC KVSNKALPAP IEKTISKAKG |
| 121 | QPREPQVYTL PPSRDELTKN QVSLTCLVKG FYPSDIAVEW ESNGQPENNY KTTPPVLDSD |
| 181 | GSFFLYSKLT VDKSRWQQGN VFSCSVMHEA LHNHYTQKSL SLSPGK |

In some embodiments, the polypeptide of the invention includes a modified IgG1 Fc polypeptide, wherein the modified IgG1 Fc polypeptide of the fusion protein includes mutations at residues L234 and L235, which correspond to residues 4 and 5 of SEQ ID NO: 6 or residues 14 and 15 of SEQ ID NO: 7 shown above, and has the following amino acid sequence, where the mutated amino acid residues are boxed:

| (SEQ ID NO: 12) | |
|---|---|
| 1 | 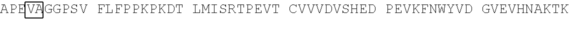 |
| 61 | PREEQYNSTY RVVSVLTVLH QDWLNGKEYK CKVSNKALPA PIEKTISKAK GQPREPQVYT |
| 121 | LPPSRDELTK NQVSLTCLVK GFYPSDIAVE WESNGQPENN YKTTPPVLDS DGSFFLYSKL |
| 181 | TVDKSRWQQG NVFSCSVMHE ALHNHYTQKS LSLSPGK |

In some embodiments, the polypeptide of the invention includes a hinge region coupled to the N-terminus of a modified IgG1 Fc polypeptide, the modified IgG1 Fc polypeptide of the polypeptide includes mutations at residues L234 and L235, which correspond to residues 4 and 5 of SEQ ID NO: 6 or residues 14 and 15 of SEQ ID NO: 7 shown above, and the fusion protein includes at least the following amino acid sequence, where the mutated amino acid residues are boxed:

| (SEQ ID NO: 13) | |
|---|---|
| 1 | 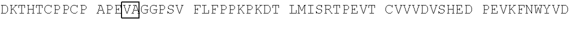 |
| 61 | GVEVHNAKTK PREEQYNSTY RVVSVLTVLH QDWLNGKEYK CKVSNKALPA PIEKTISKAK |
| 121 | GQPREPQVYT LPPSRDELTK NQVSLTCLVK GFYPSDIAVE WESNGQPENN YKTTPPVLDS |
| 181 | DGSFFLYSKL TVDKSRWQQG NVFSCSVMHE ALHNHYTQKS LSLSPGK |

In some embodiments the polypeptide of the invention includes a modified IgG1 Fc polypeptide, the modified IgG1 Fc polypeptide of the fusion protein includes a deletion at residue G236 and mutations at residues L234 and L235, and the fusion protein includes at least the following amino acid sequence, where the mutated amino acid residues are boxed:

| (SEQ ID NO: 14) | |
|---|---|
| 1 | 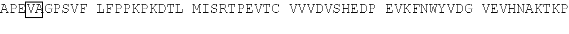 |
| 61 | REEQYNSTYR VVSVLTVLHQ DWLNGKEYKC KVSNKALPAP IEKTISKAKG QPREPQVYTL |
| 121 | PPSRDELTKN QVSLTCLVKG FYPSDIAVEW ESNGQPENNY KTTPPVLDSD GSFFLYSKLT |
| 181 | VDKSRWQQGN VFSCSVMHEA LHNHYTQKSL SLSPGK |

In some embodiments, the polypeptide of the invention includes a hinge region coupled to the N-terminus of a modified IgG1 Fc polypeptide, the modified IgG1 Fc polypeptide of the fusion protein includes a deletion at residue G236 and mutations at residues L234 and L235, and has the following amino acid sequence, where the mutated amino acid residues are boxed:

| (SEQ ID NO: 15) | |
|---|---|
| 1 | 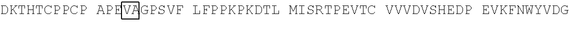 |
| 61 | VEVHNAKTKP REEQYNSTYR VVSVLTVLHQ DWLNGKEYKC KVSNKALPAP IEKTISKAKG |
| 121 | QPREPQVYTL PPSRDELTKN QVSLTCLVKG FYPSDIAVEW ESNGQPENNY KTTPPVLDSD |
| 181 | GSFFLYSKLT VDKSRWQQGN VFSCSVMHEA LHNHYTQKSL SLSPGK |

In some embodiments, the polypeptide of the invention includes a modified IgG1 Fc polypeptide, the modified IgG1 Fc polypeptide of the fusion protein includes a deletion at residue G236 and mutations at residues L234, L235, M252, T256, and M428, and the fusion protein includes at least the following amino acid sequence, where the mutated amino acid residues are boxed:

| (SEQ ID NO: 16) | |
|---|---|
| 1 | 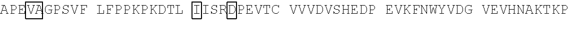 |
| 61 | REEQYNSTYR VVSVLTVLHQ DWLNGKEYKC KVSNKALPAP IEKTISKAKG QPREPQVYTL |
| 121 | PPSRDELTKN QVSLTCLVKG FYPSDIAVEW ESNGQPENNY KTTPPVLDSD GSFFLYSKLT |
| 181 | 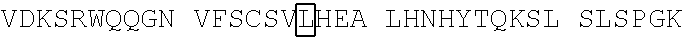 |

In some embodiments, polypeptide of the invention includes a hinge region coupled to the N-terminus of a modified IgG1 Fc polypeptide, the modified IgG1 Fc polypeptide of the fusion protein includes a deletion at residue G236 and mutations at residues L234, L235, M252, T256, and M428, and has the following amino acid sequence, where the mutated amino acid residues are boxed:

| (SEQ ID NO: 17) | |
|---|---|
| 1 | 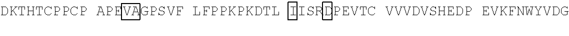 |
| 61 | VEVHNAKTKP REEQYNSTYR VVSVLTVLHQ DWLNGKEYKC KVSNKALPAP IEKTISKAKG |
| 121 | QPREPQVYTL PPSRDELTKN QVSLTCLVKG FYPSDIAVEW ESNGQPENNY KTTPPVLDSD |
| 181 | 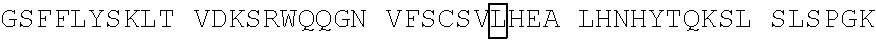 |

In some embodiments, the polypeptide of the invention includes a modified IgG1 Fc polypeptide, the modified IgG1 Fc polypeptide of the fusion protein includes a modified human IgG1 Fc polypeptide sequence that is at least 50%, 60%, 65%, 70%, 75%, 80%, 85%, 90%, 91%, 92%, 93%, 94%, 95%, 96%, 97%, 98% or 99% identical to the amino acid sequence of SEQ ID NO: 8, 9, 10, 11, 12, 13, 14, 15, 16, or 17.

In some embodiments, the polypeptide of the invention includes an Fc polypeptide, the Fc polypeptide of the fusion protein includes a human IgG2 Fc polypeptide sequence having the following amino acid sequence:

| (SEQ ID NO: 18) | |
|---|---|
| 1 | APPVAGPSVF LFPPKPKDTL MISRTPEVTC VVVDVSHEDP EVQFNWYVDG VEVHNAKTKP |
| 61 | REEQFNSTFR VVSVLTVVHQ DWLNGKEYKC KVSNKGLPAP IEKTISKTKG QPREPQVYTL |
| 121 | PPSREEMTKN QVSLTCLVKG FYPSDIAVEW ESNGQPENNY KTTPPMLDSD GSFFLYSKLT |
| 181 | VDKSRWQQGN VFSCSVMHEA LHNHYTQKSL SLSPGK |

In some embodiments where the fusion protein of the invention includes an Fc polypeptide, the Fc polypeptide of the fusion protein includes a human IgG2 Fc polypeptide sequence that is at least 50%, 60%, 65%, 70%, 75%, 80%, 85%, 90%, 91%, 92%, 93%, 94%, 95%, 96%, 97%, 98% or 99% identical to the amino acid sequence of SEQ ID NO: 18.

In some embodiments, the polypeptide of the invention includes a modified IgG2 Fc polypeptide, the modified IgG2 Fc polypeptide of the fusion protein includes a deletion at residue G236 and mutations at residues M252, T256, and M428, and has the following amino acid sequence, where the mutated amino acid residues are boxed:

| (SEQ ID NO: 19) | |
|---|---|
| 1 | 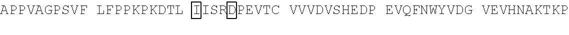 |
| 61 | REEQFNSTFR VVSVLTVVHQ DWLNGKEYKC KVSNKGLPAP IEKTISKTKG QPREPQVYTL |
| 121 | PPSREEMTKN QVSLTCLVKG FYPSDIAVEW ESNGQPENNY KTTPPMLDSD GSFFLYSKLT |
| 181 | 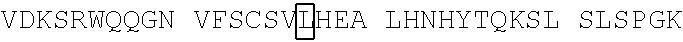 |

In some embodiments, the polypeptide of the invention includes an Fc polypeptide, the Fc polypeptide of the fusion protein includes a human IgG3 Fc polypeptide sequence having the following amino acid sequence:

| (SEQ ID NO: 20) | |
|---|---|
| 1 | APELLGGPSV FLFPPKPKDT LMISRTPEVT CVVVDVSHED PEVQFKWYVD GVEVHNAKTK |
| 61 | PREEQYNSTF RVVSVLTVLH QDWLNGKEYK CKVSNKALPA PIEKTISKTK GQPREPQVYT |
| 121 | LPPSREEMTK NQVSLTCLVK GFYPSDIAVE WESSGQPENN YNTTPPMLDS DGSFFLYSKL |
| 181 | TVDKSRWQQG NIFSCSVMHE ALHNRFTQKS LSLSPGK |

In some embodiments, the polypeptide of the invention includes an Fc polypeptide, the Fc polypeptide of the fusion protein includes a human IgG3 Fc polypeptide sequence that is at least 50%, 60%, 65%, 70%, 75%, 80%, 85%, 90%, 91%, 92%, 93%, 94%, 95%, 96%, 97%, 98% or 99% identical to the amino acid sequence of SEQ ID NO: 20.

In some embodiments, the polypeptide of the invention includes a modified IgG3 Fc polypeptide, the modified IgG3 Fc polypeptide of the fusion protein includes mutations at residues M252, T256, and M428, and has the following amino acid sequence, where the mutated amino acid residues are boxed:

| (SEQ ID NO: 21) | |
|---|---|
| 1 | 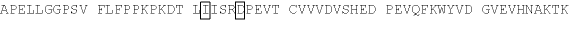 |
| 61 | PREEQYNSTF RVVSVLTVLH QDWLNGKEYK CKVSNKALPA PIEKTISKTK GQPREPQVYT |
| 121 | LPPSREEMTK NQVSLTCLVK GFYPSDIAVE WESSGQPENN YNTTPPMLDS DGSFFLYSKL |
| 181 | 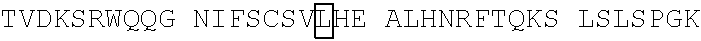 |

In some embodiments, the polypeptide of the invention includes a modified IgG3 Fc polypeptide, the modified IgG3 Fc polypeptide of the fusion protein includes a modified human IgG3 Fc polypeptide sequence where residue G236, which corresponds to residue 6 of SEQ ID NO: 20 shown above, is deleted and has the following amino acid sequence:

| (SEQ ID NO: 22) | |
|---|---|
| 1 | APELLGPSVF LFPPKPKDTL MISRTPEVTC VVVDVSHEDP EVQFKWYVDG VEVHNAKTKP |
| 61 | REEQYNSTFR VVSVLTVLHQ DWLNGKEYKC KVSNKALPAP IEKTISKTKG QPREPQVYTL |
| 121 | PPSREEMTKN QVSLTCLVKG FYPSDIAVEW ESSGQPENNY NTTPPMLDSD GSFFLYSKLT |
| 181 | VDKSRWQQGN IFSCSVMHEA LHNRFTQKSL SLSPGK |

In some embodiments, the polypeptide of the invention includes a modified IgG3 Fc polypeptide, the modified IgG3 Fc polypeptide of the fusion protein includes mutations at residues L234 and L235, which correspond to residues 4 and 5 of SEQ ID NO: 20 shown above, and has the following amino acid sequence, where the mutated amino acid residues are boxed:

| (SEQ ID NO: 23) | |
|---|---|
| 1 | 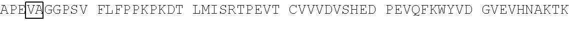 |
| 61 | PREEQYNSTF RVVSVLTVLH QDWLNGKEYK CKVSNKALPA PIEKTISKTK GQPREPQVYT |
| 121 | LPPSREEMTK NQVSLTCLVK GFYPSDIAVE WESSGQPENN YNTTPPMLDS DGSFFLYSKL |
| 181 | TVDKSRWQQG NIFSCSVMHE ALHNRFTQKS LSLSPGK |

In some embodiments where the fusion protein of the invention includes a modified IgG3 Fc polypeptide, the modified IgG3 Fc polypeptide of the fusion protein includes a deletion at residue G236 and mutations at residues L234 and L235 and has the following amino acid sequence:

| (SEQ ID NO: 24) | |
|---|---|
| 1 | 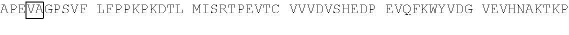 |
| 61 | REEQYNSTFR VVSVLTVLHQ DWLNGKEYKC KVSNKALPAP IEKTISKTKG QPREPQVYTL |
| 121 | PPSREEMTKN QVSLTCLVKG FYPSDIAVEW ESSGQPENNY NTTPPMLDSD GSFFLYSKLT |
| 181 | VDKSRWQQGN IFSCSVMHEA LHNRFTQKSL SLSPGK |

In some embodiments, the polypeptide of the invention includes a modified IgG3 Fc polypeptide, the modified IgG3 Fc polypeptide of the fusion protein includes a deletion at residue G236 and mutations at residues L234, L235, M252, T256, and M428, and has the following amino acid sequence:

| (SEQ ID NO: 25) | |
|---|---|
| 1 | 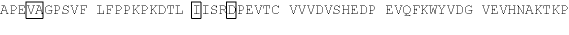 |
| 61 | REEQYNSTFR VVSVLTVLHQ DWLNGKEYKC KVSNKALPAP IEKTISKTKG QPREPQVYTL |
| 121 | PPSREEMTKN QVSLTCLVKG FYPSDIAVEW ESSGQPENNY NTTPPMLDSD GSFFLYSKLT |
| 181 | 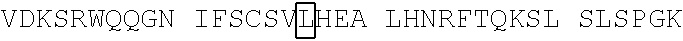 |

In some embodiments, the polypeptide of the invention includes a modified IgG3 Fc polypeptide, the modified IgG3 Fc polypeptide of the fusion protein includes a modified human IgG3 Fc polypeptide sequence that is at least 50%, 60%, 65%, 70%, 75%, 80%, 85%, 90%, 91%, 92%, 93%, 94%, 95%, 96%, 97%, 98% or 99% identical to the amino acid sequence of SEQ ID NO: 21, 22, 23, 24, or 25.

In some embodiments, the polypeptide of the invention includes an Fc polypeptide, the Fc polypeptide of the fusion protein includes a human IgG4 Fc polypeptide sequence having the following amino acid sequence:

| (SEQ ID NO: 26) | |
|---|---|
| 1 | APEFLGGPSV FLFPPKPKDT LMISRTPEVT CVVVDVSQED PEVQFNWYVD GVEVHNAKTK |
| 61 | PREEQFNSTY RVVSVLTVLH QDWLNGKEYK CKVSNKGLPS SIEKTISKAK GQPREPQVYT |
| 121 | LPPSQEEMTK NQVSLTCLVK GFYPSDIAVE WESNGQPENN YKTTPPVLDS DGSFFLYSRL |
| 181 | TVDKSRWQEG NVFSCSVMHE ALHNHYTQKS LSLSLGK |

In some embodiments, the polypeptide of the invention includes an Fc polypeptide, the Fc polypeptide of the fusion protein includes a hinge region coupled to the N-terminus of the Fc polypeptide of the fusion protein, where the Fc polypeptide includes a human IgG4 Fc polypeptide sequence having the following amino acid sequence:

| (SEQ ID NO: 27) | |
|---|---|
| 1 | ESKYGPPCPS CPAPEFLGGP SVFLFPPKPK DTLMISRTPE VTCVVVDVSQ EDPEVQFNWY |
| 61 | VDGVEVHNAK TKPREEQFNS TYRVVSVLTV LHQDWLNGKE YKCKVSNKGL PSSIEKTISK |
| 121 | AKGQPREPQV YTLPPSQEEM TKNQVSLTCL VKGFYPSDIA VEWESNGQPE NNYKTTPPVL |
| 181 | DSDGSFFLYS RLTVDKSRWQ EGNVFSCSVM HEALHNHYTQ KSLSLSLGK |

In some embodiments, where the fusion protein of the invention includes an Fc polypeptide, the Fc polypeptide of the fusion protein includes a human IgG4 Fc polypeptide sequence that is at least 50%, 60%, 65%, 70%, 75%, 80%, 85%, 90%, 91%, 92%, 93%, 94%, 95%, 96%, 97%, 98% or 99% identical to the amino acid sequence of SEQ ID NO: 26 or 27.

In some embodiments where the fusion protein of the invention includes a modified IgG4 Fc polypeptide, the modified IgG4 Fc polypeptide of the fusion protein includes mutations at residues M252, T256, and M428, which correspond to residues 22, 26, and 19 of SEQ ID NO: 26 or residues 34, 38, and 210 of SEQ ID NO: 27 shown above, and has the following amino acid sequence, where the mutated amino acid residues are boxed:

| (SEQ ID NO: 28) | |
|---|---|
| 1 | 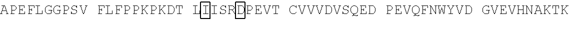 |
| 61 | PREEQFNSTY RVVSVLTVLH QDWLNGKEYK CKVSNKGLPS SIEKTISKAK GQPREPQVYT |
| 121 | LPPSQEEMTK NQVSLTCLVK GFYPSDIAVE WESNGQPENN YKTTPPVLDS DGSFFLYSRL |
| 181 | 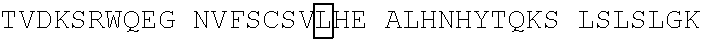 |

In some embodiments, the polypeptide of the invention includes a hinge region coupled to the N-terminus of a modified IgG4 Fc polypeptide, the modified IgG4 Fc polypeptide of the fusion protein includes mutations at residues M252, T256, and M428, which correspond to residues 22, 26, and 197 of SEQ ID NO: 26 or residues 34, 38, and 210 of SEQ ID NO: 27 shown above, and the fusion protein includes at least the following amino acid sequence, where the mutated amino acid residues are boxed:

| (SEQ ID NO: 29) | |
|---|---|
| 1 | 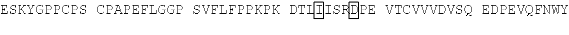 |
| 61 | VDGVEVHNAK TKPREEQFNS TYRVVSVLTV LHQDWLNGKE YKCKVSNKGL PSSIEKTISK |
| 121 | AKGQPREPQV YTLPPSQEEM TKNQVSLTCL VKGFYPSDIA VEWESNGQPE NNYKTTPPVL |
| 181 | 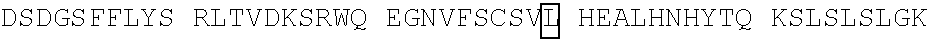 |

In some embodiments, the polypeptide of the invention includes a modified IgG4 Fc polypeptide, the modified IgG4 Fc polypeptide of the fusion protein includes a modified human IgG4 Fc polypeptide sequence where residue G236, which corresponds to residue 6 of SEQ ID NO: 26 or residue 19 of SEQ ID NO: 27 shown above, is deleted and has the following amino acid sequence:

| (SEQ ID NO: 30) | |
|---|---|
| 1 | APEFLGPSVF LFPPKPKDTL MISRTPEVTC VVVDVSQEDP EVQFNWYVDG VEVHNAKTKP |
| 61 | REEQFNSTYR VVSVLTVLHQ DWLNGKEYKC KVSNKGLPSS IEKTISKAKG QPREPQVYTL |
| 121 | PPSQEEMTKN QVSLTCLVKG FYPSDIAVEW ESNGQPENNY KTTPPVLDSD GSFFLYSRLT |
| 181 | VDKSRWQEGN VFSCSVMHEA LHNHYTQKSL SLSLGK |

In some embodiments, the polypeptide of the invention includes a hinge region coupled to the N-terminus of a modified IgG4 Fc polypeptide, the modified IgG4 Fc polypeptide of the fusion protein includes a modified human IgG4 Fc polypeptide sequence where residue G236, which corresponds to residue 6 of SEQ ID NO: 26 or residue 19 of SEQ ID NO: 27 shown above, is deleted, and the fusion protein includes at least the following amino acid sequence:

| (SEQ ID NO: 31) | |
|---|---|
| 1 | ESKYGPPCPS CPAPEFLGPS VFLFPPKPKD TLMISRTPEV TCVVVDVSQE DPEVQFNWYV |
| 61 | DGVEVHNAKT KPREEQFNST YRVVSVLTVL HQDWLNGKEY KCKVSNKGLP SSIEKTISKA |
| 121 | KGQPREPQVY TLPPSQEEMT KNQVSLTCLV KGFYPSDIAV EWESNGQPEN NYKTTPPVLD |
| 181 | SDGSFFLYSR LTVDKSRWQE GNVFSCSVMH EALHNHYTQK SLSLSLGK |

In some embodiments, the polypeptide of the invention includes a modified IgG4 Fc polypeptide, the modified IgG4 Fc polypeptide of the fusion protein includes a mutation at residue L235, which corresponds to residue 5 of SEQ ID NO: 26 or residue 17 of SEQ ID NO: 27 shown above, and has the following amino acid sequence, where the mutated amino acid residue is boxed:

| (SEQ ID NO: 32) | |
|---|---|
| 1 | 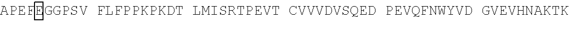 |
| 61 | PREEQFNSTY RVVSVLTVLH QDWLNGKEYK CKVSNKGLPS SIEKTISKAK GQPREPQVYT |
| 121 | LPPSQEEMTK NQVSLTCLVK GFYPSDIAVE WESNGQPENN YKTTPPVLDS DGSFFLYSRL |
| 181 | TVDKSRWQEG NVFSCSVMHE ALHNHYTQKS LSLSLGK |

In some embodiments, the polypeptide of the invention includes a hinge region coupled to the N-terminus of a modified IgG4 Fc polypeptide, the modified IgG4 Fc polypeptide of the fusion protein includes a mutation at residue L235, which corresponds to residue 5 of SEQ ID NO: 26 or residue 17 of SEQ ID NO: 27 shown above, and the fusion protein includes at least the following amino acid sequence, where the mutated amino acid residue is boxed:

| (SEQ ID NO: 33) | |
|---|---|
| 1 | 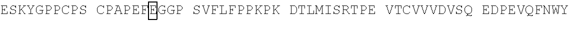 |
| 61 | VDGVEVHNAK TKPREEQFNS TYRVVSVLTV LHQDWLNGKE YKCKVSNKGL PSSIEKTISK |
| 121 | AKGQPREPQV YTLPPSQEEM TKNQVSLTCL VKGFYPSDIA VEWESNGQPE NNYKTTPPVL |
| 181 | DSDGSFFLYS RLTVDKSRWQ EGNVFSCSVM HEALHNHYTQ KSLSLSLGK |

In some embodiments, the polypeptide of the invention includes a modified IgG4 Fc polypeptide, the modified IgG4 Fc polypeptide of the fusion protein includes mutations at residues L234 and L235, which correspond to residues 4 and 5 of SEQ ID NO: 26 or residues 16 and 17 of SEQ ID NO: 27 shown above, and has the following amino acid sequence, where the mutated amino acid residues are boxed:

| (SEQ ID NO: 34) | |
|---|---|
| 1 | 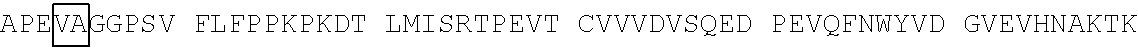 |
| 61 | PREEQFNSTY RVVSVLTVLH QDWLNGKEYK CKVSNKGLPS SIEKTISKAK GQPREPQVYT |
| 121 | LPPSQEEMTK NQVSLTCLVK GFYPSDIAVE WESNGQPENN YKTTPPVLDS DGSFFLYSRL |
| 181 | TVDKSRWQEG NVFSCSVMHE ALHNHYTQKS LSLSLGK |

In some embodiments, the polypeptide of the invention includes a hinge region coupled to the N-terminus of a modified IgG4 Fc polypeptide, the modified IgG4 Fc polypeptide of the fusion protein includes mutations at residues L234 and L235, which correspond to residues 4 and 5 of SEQ ID NO: 26 or residues 16 and 17 of SEQ ID NO: 27 shown above, and the fusion protein includes at least the following amino acid sequence, where the mutated amino acid residues are boxed:

| (SEQ ID NO: 35) | |
|---|---|
| 1 | 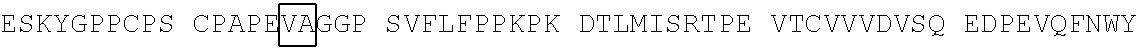 |
| 61 | VDGVEVHNAK TKPREEQFNS TYRVVSVLTV LHQDWLNGKE YKCKVSNKGL PSSIEKTISK |
| 121 | AKGQPREPQV YTLPPSQEEM TKNQVSLTCL VKGFYPSDIA VEWESNGQPE NNYKTTPPVL |
| 181 | DSDGSFFLYS RLTVDKSRWQ EGNVFSCSVM HEALHNHYTQ KSLSLSLGK |

In some embodiments, the polypeptide of the invention includes a modified IgG4 Fc polypeptide, the modified IgG4 Fc polypeptide of the fusion protein includes a mutation at residue S228, which corresponds to residue 10 of SEQ ID NO: 27 shown above, and has the following amino acid sequence, where the mutated amino acid residue is boxed:

| (SEQ ID NO: 36) | |
|---|---|
| 1 | 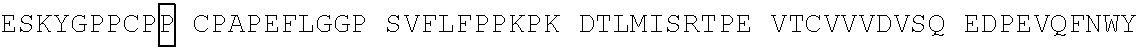 |
| 61 | VDGVEVHNAK TKPREEQFNS TYRVVSVLTV LHQDWLNGKE YKCKVSNKGL PSSIEKTISK |
| 121 | AKGQPREPQV YTLPPSQEEM TKNQVSLTCL VKGFYPSDIA VEWESNGQPE NNYKTTPPVL |
| 181 | DSDGSFFLYS RLTVDKSRWQ EGNVFSCSVM HEALHNHYTQ KSLSLSLGK |

In some embodiments, the polypeptide of the invention includes a hinge region coupled to the N-terminus of a modified IgG4 Fc polypeptide, the modified IgG4 Fc polypeptide of the fusion protein includes mutations at residues S228 and L235, which correspond to residues 10 and 17 of SEQ ID NO: 27 shown above, and the fusion protein includes at least the following amino acid sequence, where the mutated amino acid residues are boxed:

| (SEQ ID NO: 37) | |
|---|---|
| 1 | 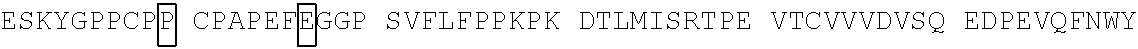 |
| 61 | VDGVEVHNAK TKPREEQFNS TYRVVSVLTV LHQDWLNGKE YKCKVSNKGL PSSIEKTISK |
| 121 | AKGQPREPQV YTLPPSQEEM TKNQVSLTCL VKGFYPSDIA VEWESNGQPE NNYKTTPPVL |
| 181 | DSDGSFFLYS RLTVDKSRWQ EGNVFSCSVM HEALHNHYTQ KSLSLSLGK |

In some embodiments, the polypeptide of the invention includes a hinge region coupled to the N-terminus of a modified IgG4 Fc polypeptide, the modified IgG4 Fc polypeptide of the fusion protein includes mutations at residues S228, L235 and M252 which correspond to residues 10, 17 and 34 of SEQ ID NO: 27 shown above, and the fusion protein includes at least the following amino acid sequence, where the mutated amino acid residues are boxed:

| (SEQ ID NO: 38) | |
|---|---|
| 1 | 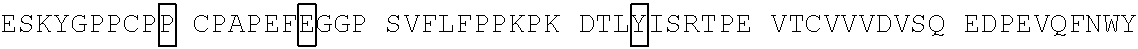 |
| 61 | VDGVEVHNAK TKPREEQFNS TYRVVSVLTV LHQDWLNGKE YKCKVSNKGL PSSIEKTISK |
| 121 | AKGQPREPQV YTLPPSQEEM TKNQVSLTCL VKGFYPSDIA VEWESNGQPE NNYKTTPPVL |
| 181 | DSDGSFFLYS RLTVDKSRWQ EGNVFSCSVM HEALHNHYTQ KSLSLSLGK |

In some embodiments, the polypeptide of the invention includes a hinge region coupled to the N-terminus of a modified IgG4 Fc polypeptide, the modified IgG4 Fc polypeptide of the fusion protein includes mutations at residues S228, L235 and M428 which correspond to residues 10, 17 and 34 of SEQ ID NO: 27 shown above, the fusion protein includes at least the following amino acid sequence, where the mutated amino acid residues are boxed:

| (SEQ ID NO: 39) | |
|---|---|
| 1 | 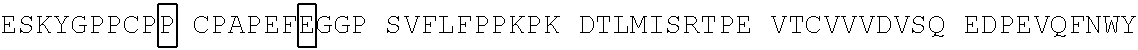 |
| 61 | VDGVEVHNAK TKPREEQFNS TYRVVSVLTV LHQDWLNGKE YKCKVSNKGL PSSIEKTISK |
| 121 | AKGQPREPQV YTLPPSQEEM TKNQVSLTCL VKGFYPSDIA VEWESNGQPE NNYKTTPPVL |
| 181 | 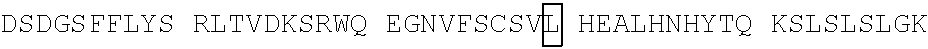 |

In some embodiments, the polypeptide of the invention includes a hinge region coupled to the N-terminus of a modified IgG4 Fc polypeptide, the modified IgG4 Fc polypeptide of the fusion protein includes mutations at residues S228, L235, M252 and M428 which correspond to residues 10, 17, 34 and 210 of SEQ ID NO: 27 shown above, the fusion protein includes at least the following amino acid sequence, where the mutated amino acid residues are boxed:

| (SEQ ID NO: 40) | |
|---|---|
| 1 | 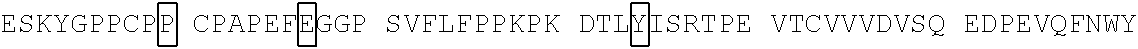 |
| 61 | VDGVEVHNAK TKPREEQFNS TYRVVSVLTV LHQDWLNGKE YKCKVSNKGL PSSIEKTISK |
| 121 | AKGQPREPQV YTLPPSQEEM TKNQVSLTCL VKGFYPSDIA VEWESNGQPE NNYKTTPPVL |
| 181 | 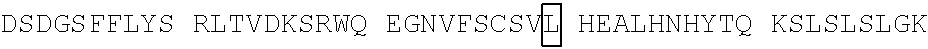 |

In some embodiments, the polypeptide of the invention includes a modified IgG4 Fc polypeptide, the modified IgG4 Fc polypeptide of the fusion protein includes mutations at residues L235, M252, T256, and M428, which correspond to residues 5, 22, 26, and 197 of SEQ ID NO: 26 or residues 17, 34, 38, and 210 of SEQ ID NO: 27 shown above, and has the following amino acid sequence, where the mutated amino acid residues are boxed:

| (SEQ ID NO: 41) | |
|---|---|
| 1 | 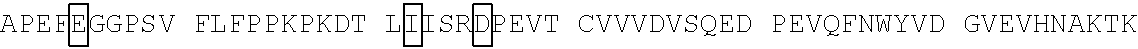 |
| 61 | PREEQFNSTY RVVSVLTVLH QDWLNGKEYK CKVSNKGLPS SIEKTISKAK GQPREPQVYT |
| 121 | LPPSQEEMTK NQVSLTCLVK GFYPSDIAVE WESNGQPENN YKTTPPVLDS DGSFFLYSRL |
| 181 | 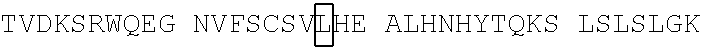 |

In some embodiments, the polypeptide of the invention includes a hinge region coupled to the N-terminus of a modified IgG4 Fc polypeptide, the modified IgG4 Fc polypeptide of the fusion protein includes mutations at residues L235, M252, T256, and M428, which correspond to residues 5, 22, 26, and 197 of SEQ ID NO: 26 or residues 17, 34, 38, and 210 of SEQ ID NO: 27 shown above, and the fusion protein includes at least the following amino acid sequence, where the mutated amino acid residues are boxed:

| (SEQ ID NO: 42) | |
|---|---|
| 1 | 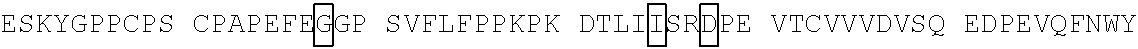 |
| 61 | VDGVEVHNAK TKPREEQFNS TYRVVSVLTV LHQDWLNGKE YKCKVSNKGL PSSIEKTISK |
| 121 | AKGQPREPQV YTLPPSQEEM TKNQVSLTCL VKGFYPSDIA VEWESNGQPE NNYKTTPPVL |
| 181 | 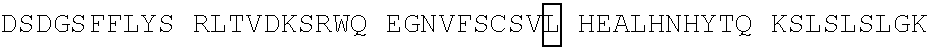 |

In some embodiments, the polypeptide of the invention includes a hinge region coupled to the N-terminus of a modified IgG4 Fc polypeptide, the modified IgG4 Fc polypeptide of the fusion protein includes mutations at residues S228, L235, M252, T256, and M428, which correspond to residues 10, 17, 34, 38, and 210 of SEQ ID NO: 27 shown above, and the fusion protein includes at least the following amino acid sequence, where the mutated amino acid residues are boxed:

| (SEQ ID NO: 43) | |
|---|---|
| 1 | 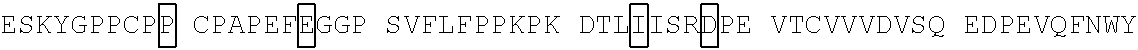 |
| 61 | VDGVEVHNAK TKPREEQFNS TYRVVSVLTV LHQDWLNGKE YKCKVSNKGL PSSIEKTISK |
| 121 | AKGQPREPQV YTLPPSQEEM TKNQVSLTCL VKGFYPSDIA VEWESNGQPE NNYKTTPPVL |
| 181 | 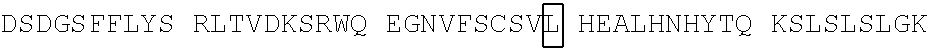 |

In some embodiments where the fusion protein of the invention includes a modified IgG4 Fc polypeptide, the modified IgG4 Fc polypeptide of the fusion protein includes a modified human IgG4 Fc polypeptide sequence that is at least 50%, 60%, 65%, 70%, 75%, 80%, 85%, 90%, 91%, 92%, 93%, 94%, 95%, 96%, 97%, 98% or 99% identical to the amino acid sequence of SEQ ID NO: 28, 29, 30, 31, 32, 33, 34, 35, 36, 37, 38, 39, 40, 41, 42 or 43.

For example, the human immunoglobulin Fc polypeptide or a polypeptide that is derived from an immunoglobulin Fc polypeptide is a modified human IgG4 Fc polypeptide that has the sequence of SEQ ID NO: 28, 29, 30, 31, 32, 33, 34, 35, 36, 37, 38, 39, 40, 41, 42 or 43.

In some embodiments, the polypeptide of the invention includes an Fc polypeptide that is derived from a modified human IgG4 Fc polypeptide, wherein the modified human IgG4 Fc polypeptide comprises mutations at positions S228, L235, M252, T256, and M428.

In some embodiments, the polypeptide of the invention includes a modified human IgG4 Fc polypeptide, wherein the modified human IgG4 Fc polypeptide comprises the amino acid sequence of SEQ ID NOs: 27, 36 or 37, wherein the modified human IgG4 Fc polypeptide comprises a mutation at position M252 (residue 34 of SEQ ID NO: 27) and/or at position, M428, (residue 210 of SEQ ID NO: 27).

In an embodiment, the modified human IgG4 Fc polypeptide further comprises a mutations at position T256 (residue 38 of SEQ ID NO: 27).

In some embodiments, the polypeptide of the invention includes an Fc polypeptide, the Fc polypeptide of the fusion protein includes a human IgM Fc polypeptide sequence having the following amino acid sequence:

| (SEQ ID NO: 44) | |
|---|---|
| 1 | IAELPPKVSV FVPPRDGFFG NPRKSKLICQ ATGFSPRQIQ VSWLREGKQV GSGVTTDQVQ |
| 61 | AEAKESGPTT YKVTSTLTIK ESDWLGQSMF TCRVDHRGLT FQQNASSMCV PDQDTAIRVF |
| 121 | AIPPSFASIF LTKSTKLTCL VTDLTTYDSV TISWTRQNGE AVKTHTNISE SHPNATFSAV |
| 181 | GEASICEDDW NSGERFTCTV THTDLPSPLK QTISRPKG |

In an embodiment, the human immunoglobulin Fc polypeptide or a polypeptide that is derived from an immunoglobulin Fc polypeptide is a modified human IgG4 Fc polypeptide.

In an embodiment, each monomer of the dimeric protein has the sequence of SEQ ID No: 45. As shown below, AAT polypeptide portion of the fusion protein is underlined (SEQ ID NO: 1) and the IgG-Fc polypeptide portion of the fusion protein is italicized (SEQ ID NO: 6).

(SEQ ID NO: 45)
EDPQGDAAQKTDTSHHDQDHPTFNKITPNLAEFAFSLYRQLAHQSNSTNI
FFSPVSIATAFAMLSLGTKADTHDEILEGLNFNLTEIPEAQIHEGFQELL
RTLNQPDSQLQLTTGNGLFLSEGLKLVDKFLEDVKKLYHSEAFTVNFGDT
EEAKKQINDYVEKGTQGKIVDLVKELDRDTVFALVNYIFFKGKWERPFEV
KDTEEEDFHVDQVTTVKVPMMKRLGMFNIQHCKKLSSWVLLMKYLGNATA
IFFLPDEGKLQHLENELTHDIITKFLENEDRRSASLHLPKLSITGTYDLK
SVLGQLGITKVFSNGADLSGVTEEAPLKLSKAVHKAVLTIDEKGTEAAGA
MFLEAIPMSIPPEVKFNKPFVFLMIEQNTKSPLFMGKVVNPTQKEPKSCD
KTHTCPPCPAPELLGGPSVFLFPPKPKDTLMISRTPEVTCVVVDVSH
EDPEVKFNWYVDGVEVHNAKTEPREEQYNSTYRVVSVLTVLHQDWLN
GKEYKCKVSNEALPAPIEKTISKAKGQPREPQVYTLPPSRDELTKNQ
VSLTCLVKGFYPSDIAVEWESNGQPENNYKTTPPVLDSDGSFFLYSK
LTVDKSRWQQGNVFSCSVMHEALHNHYTQKSLSLSPGK

In an embodiment, each monomer of the dimeric protein has the sequence of SEQ ID No: 46. As shown below, AAT polypeptide portion of the fusion protein is underlined (SEQ ID NO: 1) and the IgG-Fc polypeptide portion of the fusion protein is italicized (SEQ ID NO: 18).

(SEQ ID NO: 46)
EDPQGDAAQKTDTSHHDQDHPTFNKITPNLAEFAFSLYRQLAHQSNSTNI
FFSPVSIATAFAMLSLGTKADTHDEILEGLNFNLTEIPEAQIHEGFQELL
RTLNQPDSQLQLTTGNGLFLSEGLKLVDKFLEDVKKLYHSEAFTVNFGDT
EEAKKQINDYVEKGTQGKIVDLVKELDRDTVFALVNYIFFKGKWERPFEV
KDTEEEDFHVDQVTTVKVPMMKRLGMFNIQHCKKLSSWVLLMKYLGNATA
IFFLPDEGKLQHLENELTHDIITKFLENEDRRSASLHLPKLSITGTYDLK
SVLGQLGITKVFSNGADLSGVTEEAPLKLSKAVHKAVLTIDEKGTEAAGA
MFLEAIPMSIPPEVKFNKPFVFLMIEQNTKSPLFMGKVVNPTQKERK
CCVECPPCPAPPVAGPSVFLFPPKPKDTLMISRTPEVTCVVVDVSHE
DPEVQFNWYVDGVEVHNAKTEPREEQFNSTFRVVSVLTVVHQDWLNG
KEYKCKVSNKGLPAPIEKTISKTKGQPREPQVYTLPPSREEMTKNQV
SLTCLVKGFYPSDIAVEWESNGQPENNYKTTPPMLDSDGSFFLYSKL
TVDKSRWQQGNVFSCSVMHEALHNHYTQKSLSLSPGK

In an embodiment each monomer of the dimeric protein has the sequence of SEQ ID No: 47. As shown below, AAT polypeptide portion of the fusion protein is underlined (SEQ ID NO: 48), the IgG-Fc polypeptide portion of the fusion protein is italicized (SEQ ID NO: 6), and the Met351Glu mutation is boxed, and the Met358Leu mutation is shaded in grey.

(SEQ ID NO: 47)
EDPQGDAAQKTDTSHHDQDHPTFNKITPNLAEFAFSLYRQLAHQSNSTNIFFSPVSIATAFA
MLSLGTKADTHDEILEGLNFNLTEIPEAQIHEGFQELLRTLNQPDSQLQLTTGNGLFLSEGL
KLVDKFLEDVKKLYHSEAFTVNFGDTEEAKKQINDYVEKGTQGKIVDLVKELDRDTVFALVN
YIFFKGKWERPFEVKDTEEEDFHVDQVTTVKVPMMKRLGMFNIQHCKKLSSWVLLMKYLGNA
TAIFFLPDEGKLQHLENELTHDIITKFLENEDRRSASLHLPKLSITGTYDLKSVLGQLGITK

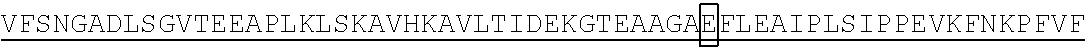

LMIEQNTKSPLFMGKVVNPTQKEPKSCDKTHTCPPCPAPELLGGPSVFLFPPKPKDTLMISR
TPEVTCVVVDVSHEDPEVKFNWYVDGVEVHNAKTKPREEQYNSTYRVVSVLTVLHODWLNGK
EYKCKVSNKALPAPIEKTISKAKGOPREPOVYTLPPSRDELTKNOVSLTCLVKGFYPSDIAV
EWESNGQPENNYKTTPPVLDSDGSFFLYSKLTVDKSRWQQGNVFSCSVMHEALHNHYTCKSL
SLSPGK

In an embodiment each monomer of the dimeric protein has the sequence of SEQ ID No: 49. As shown below, AAT polypeptide portion of the fusion protein is underlined (SEQ ID NO: 48), the IgG-Fc polypeptide portion of the fusion protein is italicized (SEQ ID NO: 18), the Met351Glu mutation is boxed, and the Met358Leu mutation is shaded in grey.

(SEQ ID NO: 49)
EDPQGDAAQKTDTSHHDQDHPTFNKITPNLAEFAFSLYRQLAHQSNSTNIFFSPVSIATAFA
MLSLGTKADTHDEILEGLNFNLTEIPEAQIHEGFQELLRTLNQPDSQLQLTTGNGLFLSEGL
KLVDKFLEDVKKLYHSEAFTVNFGDTEEAKKQINDYVEKGTQGKIVDLVKELDRDTVFALVN
YIFFKGKWERPFEVKDTEEEDFHVDQVTTVKVPMMKRLGMFNIQHCKKLSSWVLLMKYLGNA
TAIFFLPDEGKLQHLENELTHDIITKFLENEDRRSASLHLPKLSITGTYDLKSVLGQLGITK

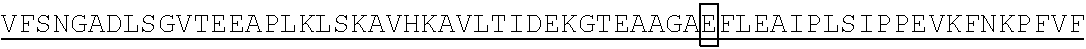

LMIEQNTKSPLFMGKVVNPTQKERKCCVECPPCPAPPVAGPSVFLFPPKPKDTLMISRTPEV
TCVVVDVSHEDPEVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKGFYPSDIAVEWES
NGQPENNYKTTPPMLDSDGSFFLYSKLTVDKSRWQQGNVFSCSVMHEALHNHYTQKSLSLSP
GK

In an embodiment each monomer of the dimeric protein has the sequence of SEQ ID No: 50. As shown below, AAT polypeptide portion of the fusion protein is underlined (SEQ ID NO: 51), the IgG-Fc polypeptide portion of the fusion protein is italicized (SEQ ID NO: 18), the Met351Leu mutation is bold and italicized, and the Met358Leu mutation is bold and italicized.

(SEQ ID NO: 50)
EDPQGDAAQKTDTSHHDQDHPTFNKITPNLAEFAFSLYRQLAHQSN
STNIFFSPVSIATAFAMLSLGTKADTHDEILEGLNFNLTEIPEAQI
HEGFQELLRTLNQPDSQLQLTTGNGLFLSEGLKLVDKFLEDVKKLY
HSEAFTVNFGDTEEAKKQINDYVEKGTQGKIVDLVKELDRDTVFAL
VNYIFFKGKWERPFEVKDTEEEDFHVDQVTTVKVPMMKRLGMFNIQ
HCKKLSSWVLLMKYLGNATAIFFLPDEGKLQHLENELTHDIITKFL
ENEDRRSASLHLPKLSITGTYDLKSVLGQLGITKVFSNGADLSGVT
EEAPLKLSKAVHKAVLTIDEKGTEAAGA [symbol] FLEAIP [symbol] SIPPEVKFNK
PFVFLMIEQNTKSPLFMGKVVNPTQKERKCCVECPPCPAPPVAGPS
VFLFPPKPKDTLMISRTPEVTCVVVDVSHEDPEVQFNWYVDGVEVH
NAKTKPREEQFNSTFRVVSVLTVVHQDWLNGKEYKCKVSNKGLPAP
IEKTISKTKGQPREPQVYTLPPSREEMTKNQVSLTCLVKGFYPSDI
AVEWESNGQPENNYKTTPPMLDSDGSFFLYSKLTVDKSRWQQGNVF
SCSVMHEALHNHYTQKSLSLSPGK

In an embodiment each monomer of the dimeric protein has the sequence of SEQ ID No: 52. As shown below, AAT polypeptide portion of the fusion protein is underlined (SEQ ID NO: 1), the IgG-Fc polypeptide portion of the fusion protein is italicized (SEQ ID NO: 6).

(SEQ ID NO: 52)
EDPQGDAAQKTDTSHHDQDHPTFNKITPNLAEFAFSLYRQLAHQSN
STNIFFSPVSIATAFAMLSLGTKADTHDEILEGLNFNLTEIPEAQI
HEGFQELLRTLNQPDSQLQLTTGNGLFLSEGLKLVDKFLEDVKKLY
HSEAFTVNFGDTEEAKKQINDYVEKGTQGKIVDLVKELDRDTVFAL
VNYIFFKGKWERPFEVKDTEEEDFHVDQVTTVKVPMMKRLGMFNIQ
HCKKLSSWVLLMKYLGNATAIFFLPDEGKLQHLENELTHDIITKFL
ENEDRRSASLHLPKLSITGTYDLKSVLGQLGITKVFSNGADLSGVT
EEAPLKLSKAVHKAVLTIDEKGTEAAGAMFLEAIPMSIPPEVKFNK
PFVFLMIEQNIKSPLFMGKVVNPTQKEPKSCDKTHTCPPCPAPELL
GGPSVFLFPPKPKDTLMISRTPEVTCVVVDVSHEDPEVKFNWYVDG
VEVHNAKTKPREEQYNSTYRVVSVLTVLHQDWLNGKEYKCKVSNKA
LPAPIEKTISKAKGQPREPQVYTLPPSRDELTKNQVSLTCLVKGFY
PSDIAVEWESNGQPENNYKTTPPVLDSDGSFFLYSELTVDKSRWQQ
GNVFSCSVMHEALHNHYTQKSLSLSPGKASTGSEDPQGDAAQKTDT
SHHDQDHPTFNKITPNLAEFAFSLYRQLAHQSNSTNIFFSPVSIAT
AFAMLSLGTKADTHDEILEGLNFNLTEIPEAQIHEGFQELLRTLNQ
PDSQLQLTTGNGLFLSEGLKLVDKFLEDVKKLYHSEAFTVNFGDTE
EAKKQINDYVEKGTQGKIVDLVKELDRDTVFALVNYIFFKGKWERP
FEVKDTEEEDFHVDQVTTVKVPMMKRLGMFNIQHCKKLSSWVLLMK
YLGNATAIFFLPDEGKLQHLENELTHDIITKFLENEDRRSASLHLP
KLSITGTYDLKSVLGQLGITKVFSNGADLSGVTEEAPLKLSKAVHK
AVLTIDEKGTEAAGAMFLEAIPMSIPPEVKFNKPFVFLMIEQNTKS
PLFMGKVVNPTQK

In an embodiment each monomer of the dimeric protein has the sequence of SEQ ID No: 53. As shown below, AAT polypeptide portion of the fusion protein is underlined with Met351Glu and Met358Leu mutations indicated in boxes (SEQ ID No: 2). The IgG1-Fc polypeptide portion of the fusion protein is italicized (SEQ ID NO: 17), with deleted Gly236, and the mutations Met25211e, Thr256Asp and Met428Leu mutations indicated in boxes.

(SEQ ID NO: 53)
EDPQGDAAQKTDTSHHDQDHPTFNKITPNLAEFAFSLYRQLAHQSNSTNIFFSPVSIATAFA
MLSLGTKADTHDEILEGLNFNLTEIPEAQIHEGFQELLRTLNQPDSQLQLTTGNGLFLSEGL
KLVDKFLEDVKKLYHSEAFTVNFGDTEEAKKQINDYVEKGTQGKIVDLVKELDRDTVFALVN
YIFFKGKWERPFEVKDTEEEDFHVDQVTTVKVPMMKRLGMFNIQHCKKLSSWVLLMKYLGNA
TAIFFLPDEGKLQHLENELTHDIITKFLENEDRRSASLHLPKLSITGTYDLKSVLGQLGITK

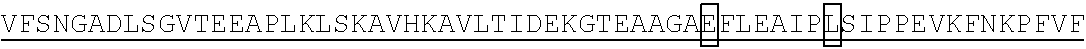

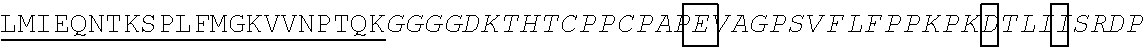

EVTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGFYPSDIAVEW

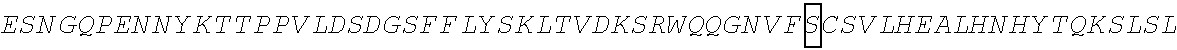

SPGK

In an embodiment each monomer of the dimeric protein has the sequence of SEQ ID No: 54. As shown below, AAT polypeptide portion of the fusion protein is underlined with Met351Glu and Met358Leu mutations indicated in boxes (SEQ ID No: 2). The IgG1-Fc polypeptide portion of the fusion protein is italicized (SEQ ID NO: 43), with Ser228Pro, Leu235G1u, Met25211e, Thr256Asp and Met428Leu mutations indicated in boxes.

(SEQ ID NO: 54)
EDPQGDAAQKTDTSHHDQDHPTFNKITPNLAEFAFSLYRQLAHQSNSTNIFFSPVSIATAFA
MLSLGTKADTHDEILEGLNFNLTEIPEAQIHEGFQELLRTLNQPDSQLQLTTGNGLFLSEGL
KLVDKFLEDVKKLYHSEAFTVNFGDTEEAKKQINDYVEKGTQGKIVDLVKELDRDTVFALVN
YIFFKGKWERPFEVKDTEEEDFHVDQVTTVKVPMMKRLGMFNIQHCKKLSSWVLLMKYLGNA
TAIFFLPDEGKLQHLENELTHDIITKFLENEDRRSASLHLPKLSITGTYDLKSVLGQLGITK

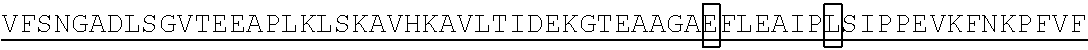

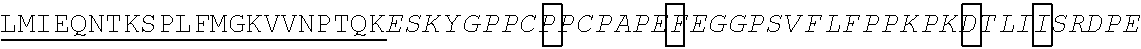

VTCVVVDVSQEDPEVCENWYVDGVEVHNAKTKPREEQFNSTYRVVSVLTVLHODWLNGKEYK
CKVSNKGLPSSIEKTISKAKGQPREPQVYTLPPSQEEMTKNQVSLTCLVKGFYPSDIAVEWE

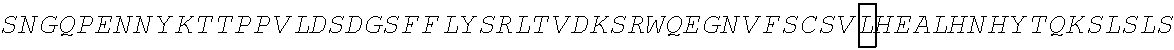

LGK

In an embodiment each monomer of the dimeric protein has the sequence of SEQ ID No: 55. As shown below, AAT polypeptide portion of the fusion protein is underlined, with AAT reactive loop sequence of SEQ ID NO: 51 with the Met351Leu mutation is bold and italicized, and the Met358Leu mutation is bold and italicized and the IgG1-Fc polypeptide portion of the fusion protein is italicized (SEQ ID NO: 7).

(SEQ ID NO: 55)
EDPQGDAAQKTDTSHHDQDHPTFNKITPNLAEFAFSLYRQLAHQSN
STNIFFSPVSIATAFAMLSLGTKADTHDEILEGLNFNLTEIPEAQI
HEGFQELLRTLNQPDSQLQLTTGNGLFLSEGLKLVDKFLEDVKKLY
HSEAFTVNFGDTEEAKKQINDYVEKGTQGKIVDLVKELDRDTVFAL
VNYIFFKGKWERPFEVKDTEEEDFHVDQVTTVKVPMMKRLGMFNIQ
HCKKLSSWVLLMKYLGNATAIFFLPDEGKLQHLENELTHDIITKFL
ENEDRRSASLHLPKLSITGTYDLKSVLGQLGITKVFSNGADLSGVT
EEAPLKLSKAVHKAVLTIDEKGTEAAGA [symbol] FLEAIP [symbol] SIPPEVKFNK
PFVFLMIEQNTKSPLFMGKVVNPTQKEPKSCDKTHTCPPCPAPELL
GGPSVFLFPPKPKDTLMISRTPEVTCVVVDVSHEDPEVKFNWYVDG
VEVHNAKTKPREEQYNSTYRVVSVLTVEHQDWLNGKEYKCKVSNKA
LPAPIEKTISKAKGQPREPQVYTLPPSRDELTKNQVSLTCLVKGFY
PSDIAVEWESNGQPENNYKTTPPVLDSDGSFFLYSKLTVDKSRWQQ
GNVFSCSVMHEALHNHYTQKSLSLSPGK

In an embodiment each monomer of the dimeric protein has the sequence of SEQ ID No: 56. As shown below, AAT polypeptide portion of the fusion protein is underlined with the Met351Leu mutation is bold and italicized, and the Met358Leu mutation is bold and italicized (SEQ ID No: 48): and the IgG4-Fc polypeptide portion of the fusion protein is italicized with mutations S228P, L235E, M252Y and M428L indicated in boxes, and a GS linker indicated in bold (SEQ ID NO: 40).

(SEQ ID NO: 56)
EDPQGDAAQKTDTSHHDQDHPTFNKITPNLAEFAFSLYRQLAHQSNSTNIFFSPVSIATAFAMLSLGTKA
DTHDEILEGLNFNLTEIPEAQIHEGFQELLRTLNQPDSQLQLTTGNGLFLSEGLKLVDKFLEDVKKLYHSEA
FTVNFGDTEEAKKQINDYVEKGTQGKIVDLVKELDRDTVFALVNYIFFKGKWERPFEVKDTEEEDFHVDQ
VTTVKVPMMKRLGMFNIQHCKKLSSWVLLMKYLGNATAIFFLPDEGKLQHLENELTHDIITKFLENEDRR
SASLHLPKLSITGTYDLKSVLGQLGITKVFSNGADLSGVTEEAPLKLSKAVHKAVLTIDEKGTEAAGAEFLEA

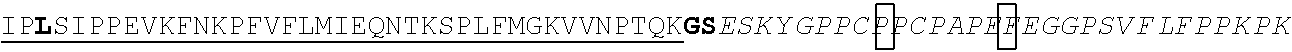

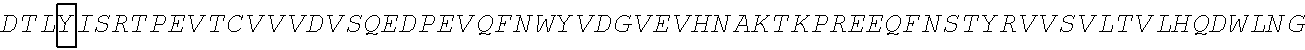

KEYKCKVSNKGLPSSIEKTISKAKGQPREPQVYTLPPSQEEMTKNQVSLTCLVKGFYPSDIAVEWESNGQP

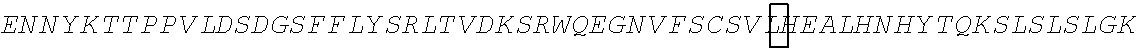

In some embodiments of the monomer of the dimeric protein of the disclosure, the IgG polypeptide portion of the monomer can be connected to the AAT polypeptide portion without a GS linker. In some embodiments, the IgG polypeptide portion of the monomer of the dimeric protein of the disclosure can be connected to the AAT polypeptide portion by covalent linkage.

The monomers of the dimeric protein may be linked to each other by disulfide bridges. In particular, the pair of human immunoglobulin Fc polypeptide or polypeptide that is derived from an immunoglobulin Fc polypeptide are so linked to form a functional Fc domain.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

### EXAMPLES

### General Methods

### Methods of Assessing Stability of an Engineered Dimeric Protein

### a) Visual Assessment

Visible particles are suitably detected using the 2.9.20. European Pharmacopoeia Monograph (Particulate Contamination: Visible Particles). The apparatus required consists of a viewing station comprising:
a matt black panel of appropriate size held in a vertical position
a non-glare white panel of appropriate size held in a vertical position next to the black panel
an adjustable lamp holder fitted with a suitable, shaded, white-light source and with a suitable light diffuser (a viewing illuminator containing two 13 W fluorescent tubes, each 525 mm in length, is suitable). The intensity of illumination at the viewing point is maintained between 2000 lux and 3750 lux.

Any adherent labels are removed from the container and the outside washed and dried. The container is gently swirled or inverted, ensuring that air bubbles are not introduced, and observed for about 5 s in front of the white panel. The procedure is repeated in front of the black panel. The presence of any particles is recorded.

The visual scores are ranked as follows:
Visual score 1: clear solution free of visible particles
Visual score 2: slight particle formation (up to ˜20 very small particles)
Visual score 3: more significant precipitation (>20 particles, including larger particles)

Whilst the particles in samples with visual score 3 are clearly detectable on casual visual assessment under normal light, samples with visual score 1 and 2 generally appear as clear solutions on the same assessment. Samples with visual scores 1 and 2 are considered to be “Pass”; samples with visual score 3 are considered to be “Fail”.

(b) Size exclusion chromatography (SEC)

### Method 1

The amount of high molecular weight species is measured using a 300×7.8 mm TSK Gel G3000 SWXL (or equivalent) size-exclusion column. The mobile phase is 250 mM potassium chloride and 200 mM potassium phosphate buffer pH 6.2, with a flow rate of 0.5 ml/min, injection volume of 4 μl (corresponding to 200 microgram of protein) and detected at 280 nm. The run time is 30 min. The results are expressed as % high molecular species (HMWS), i.e. sum of all peak areas corresponding to aggregated protein over the sum of all protein-related peaks on the chromatogram.

### Method 2

The amount of high molecular weight species is measured using a 300×7.8 mm TSK Gel G3000 SWXL (or equivalent) size-exclusion column. The mobile phase is 250 mM potassium chloride and 200 mM potassium phosphate buffer pH 6.2, with a flow rate of 0.5 ml/min, injection volume of 10 μl (corresponding to 500 microgram of protein) and detected at 280 nm. The run time is 30 min. The results are expressed as % high molecular species (HMWS), i.e. sum of all peak areas corresponding to aggregated protein over the sum of all protein-related peaks on the chromatogram.

For both methods, the increase in % HMWS means the change observed in % HMWS at a given time-point compared with the % HMWS value at time zero (i.e. immediately before incubation at the storage temperature).

(c) Sub visible particle assessment (SVP)

The number of sub visible particles per container in a liquid sample is assessed using a HIAC 9703+ Liquid Particle Counter. A blank test and particle count set are used for system suitability. Samples are degassed for 10 minutes at 75 torr before measurement and tested undiluted. Results are reported as the average of three measurements of 5 mL each.

### Example 1—Example Formulations

The following example formulation may be prepared:

### Example A

Engineered dimeric protein of the invention 50 mg/ml

| | | |
|---|---|---|
| TRIS | 3 | mM |
| Trehalose | 300 | mM |
| Proline | 100 | mM |
| Polysorbate 20 | 0.1 | mg/ml |

| | |
|---|---|
| Water for injection | qs |

pH adjusted to 7.3 using either hydrochloric acid or sodium hydroxide

| | | |
|---|---|---|
| Ionic strength | 2.6 | mM |

### Example B

Engineered dimeric protein of the invention 50 mg/ml

| | | |
|---|---|---|
| TRIS | 3 | mM |
| Trehalose | 300 | mM |
| Methionine | 2 | mM |
| Polysorbate 20 | 0.1 | mg/ml |

| | |
|---|---|
| Water for injection | qs |

pH adjusted to 7.3 using either hydrochloric acid or sodium hydroxide

| | | |
|---|---|---|
| Ionic strength | 2.6 | mM |

### Example C

Engineered dimeric protein of the invention 50 mg/ml

| | | |
|---|---|---|
| TRIS | 3 | mM |
| Trehalose | 300 | mM |
| Proline | 100 | mM |
| Methionine | 2 | mM |
| Polysorbate 20 | 0.1 | mg/ml |

| | |
|---|---|
| Water for injection | qs |

pH adjusted to 7.3 using either hydrochloric acid or sodium hydroxide

| | | |
|---|---|---|
| Ionic strength | 2.6 | mM |

### Example D

Engineered dimeric protein of the invention 50 mg/ml

| | | |
|---|---|---|
| TRIS | 5 | mM |
| Trehalose | 300 | mM |
| Proline | 100 | mM |
| Polysorbate 20 | 0.1 | mg/ml |

| | |
|---|---|
| Water for injection | qs |

pH adjusted to 7.3 using either hydrochloric acid or sodium hydroxide

| | | |
|---|---|---|
| Ionic strength | 4.3 | mM |

### Example E

Engineered dimeric protein of the invention 50 mg/ml

| | | |
|---|---|---|
| TRIS | 3 | mM |
| Trehalose | 300 | mM |
| Proline | 100 | mM |
| Poloxamer 188 | 1 | mg/ml |

| | |
|---|---|
| Water for injection | qs |

pH adjusted to 7.3 using either hydrochloric acid or sodium hydroxide

| | | |
|---|---|---|
| Ionic strength | 2.6 | mM |

### Example F

Engineered dimeric protein of the invention 50 mg/ml

| | | |
|---|---|---|
| TRIS | 5 | mM |
| Trehalose | 300 | mM |
| Proline | 100 | mM |
| Poloxamer 188 | 1 | mg/ml |

| | |
|---|---|
| Water for injection | qs |

pH adjusted to 7.3 using either hydrochloric acid or sodium hydroxide

| | | |
|---|---|---|
| Ionic strength | 4.3 | mM |

### Example G

Engineered dimeric protein of the invention 50 mg/ml

| | | |
|---|---|---|
| TRIS | 5 | mM |
| Trehalose | 150 | mM |
| Sucrose | 100 | mM |
| Proline | 100 | mM |
| Polysorbate 20 | 0.1 | mg/ml |

| | |
|---|---|
| Water for injection | qs |

pH adjusted to 7.3 using either hydrochloric acid or sodium hydroxide

| | | |
|---|---|---|
| Ionic strength | 4.3 | mM |

### Example H

Engineered dimeric protein of the invention 50 mg/ml

| | | |
|---|---|---|
| TRIS | 5 | mM |
| Trehalose | 150 | mM |
| Sucrose | 100 | mM |
| Proline | 100 | mM |
| Methionine | 2 | mM |
| Poloxamer 188 | 1 | mg/ml |

| | |
|---|---|
| Water for injection | qs |

pH adjusted to 7.3 using either hydrochloric acid or sodium hydroxide

| | | |
|---|---|---|
| Ionic strength | 4.3 | mM |

### Example I

Engineered dimeric protein of the invention 50 mg/ml

| | | |
|---|---|---|
| TRIS | 5 | mM |
| Trehalose | 150 | mM |
| Sucrose | 100 | mM |
| Glycine | 100 | mM |
| Methionine | 2 | mM |
| Poloxamer 188 | 1 | mg/ml |

| | |
|---|---|
| Water for injection | qs |

pH adjusted to 7.3 using either hydrochloric acid or sodium hydroxide

| | | |
|---|---|---|
| Ionic strength | 4.3 | mM |

### Example J

Engineered dimeric protein of the invention 50 mg/ml

| | | |
|---|---|---|
| TRIS | 5 | mM |
| Trehalose | 150 | mM |
| Sucrose | 100 | mM |
| Glycine | 100 | mM |
| Poloxamer 188 | 1 | mg/ml |

| | |
|---|---|
| Water for injection | qs |

pH adjusted to 7.3 using either hydrochloric acid or sodium hydroxide

| | | |
|---|---|---|
| Ionic strength | 4.3 | mM |

### Example K

Engineered dimeric protein of the invention 50 mg/ml

| | | |
|---|---|---|
| Sodium phosphate | 5 | mM |
| Trehalose | 150 | mM |
| Sucrose | 100 | mM |
| Proline | 100 | mM |
| Methionine | 2 | mM |
| Polysorbate 20 | 0.1 | mg/ml |

| | |
|---|---|
| Water for injection | qs |

pH adjusted to 7.3 using either hydrochloric acid or sodium hydroxide

| | | |
|---|---|---|
| Ionic strength | 11.1 | mM |

### Example L

Engineered dimeric protein of the invention 50 mg/ml

| | | |
|---|---|---|
| Sodium phosphate | 5 | mM |
| Trehalose | 150 | mM |
| Sucrose | 100 | mM |
| Proline | 100 | mM |
| Methionine | 2 | mM |
| Poloxamer 188 | 1 | mg/ml |

| | |
|---|---|
| Water for injection | qs |

pH adjusted to 7.3 using either hydrochloric acid or sodium hydroxide

| | | |
|---|---|---|
| Ionic strength | 11.1 | mM |

The stability of the formulations can be determined using a visual assessment and SEC (see General Methods) following incubation at 40° C. for 2, 4 and 8 weeks.

The stability of the formulations can be determined using a visual assessment and SEC (see General Methods) following incubation at 25° C. for 2, 4, 8, 12 and 26 weeks.

The stability of the formulations can be determined using a visual assessment and SEC (see General Methods) following incubation at 2-8° C. for 2, 4, 8, 12 and 26 weeks.

In the following Examples the engineered dimeric protein having SEQ ID NO: 56 as monomer sequence (“PROTEIN-1”) was used.

### Example 2—Effect of Ionic Strength on Storage Stability of Protein-1

The effect of ionic strength on the stability of PROTEIN-1 (50 mg/ml) was investigated by comparing a charged tonicity modifier (sodium chloride, 150 mM) with an uncharged tonicity modifier (glycerol, 300 mM).

All formulations contained polysorbate 20 (0.1 mg/mL) and either TRIS (2 mM) or sodium phosphate (2 mM) as buffer. Formulations containing TRIS were adjusted to pH 8.0 and those containing sodium phosphate were adjusted to pH 7.0. Table 1 summarises the formulations tested.

TABLE 1
Formulations of PROTEIN-1 tested. All formulations contained
PROTEIN-1 (50 mg/ml) and polysorbate 20 (0.1 mg/ml).

| | | Sodium | Sodium | | | Ionic |
|---|---|---|---|---|---|---|
| | TRIS | phosphate | chloride | Glycerol | | strength* |
| Formulation | (mM) | (mM) | (mM) | (mM) | pH | (mM) |
| 1-01 | — | 2 | 150 | — | 7.0 | 153.8 |
| 1-02 | 2 | — | 150 | — | 8.0 | 151.1 |
| 1-03 | — | 2 | — | 300 | 7.0 | 3.8 |
| 1-04 | 2 | — | — | 300 | 8.0 | 1.1 |

*Total ionic strength “I” as defined above

All formulations were stored at 25° C. for 7 weeks. Stability of PROTEIN-1 was assessed by monitoring the rate of high molecular weight species formation using SEC (Method 1), and by visual assessment, as described in the General Methods.

The rate of HMWS formation in formulations 1-01 to 1-04 is shown in Table 2, where it can be seen that the rate of HMWS formation was lowest in formulations of low ionic strength containing the uncharged tonicity modifier, glycerol (comparing formulation 1-01 with 1-03, and comparing formulation 1-02 with 1-04). The rate of HMWS formation was observed to be higher in formulations at pH 7.0 using sodium phosphate buffer, compared with the equivalent formulation at pH 8.0 using TRIS buffer (comparing formulation 1-01 with 1-02, and comparing formulation 1-03 with 1-04).

TABLE 2
Stability of PROTEIN-1 (50 mg/ml) at 25° C. for 7 weeks
in formulations 1-01 to 1-04 assessed by SEC (Method 1).

| Formulation | Increase in % HMWS |
|---|---|
| 1-01 | 3.77 |
| 1-02 | 3.65 |
| 1-03 | 1.70 |
| 1-04 | 1.56 |

### Example 3—Optimal PH for Storage Stability of Protein-1

The effect of pH on the stability of PROTEIN-1 (50 mg/ml) was investigated. All formulations contained polysorbate 20 (0.1 mg/ml), TRIS (1 mM) as buffer, and either trehalose (300 mM), a mixture of trehalose (150 mM) and mannitol (250 mM) as uncharged tonicity modifier. Some formulations contained methionine (2 mM). Table 3 summarises the formulations tested.

TABLE 3
Formulations of PROTEIN-1 tested. All formulations contained
PROTEIN-1 (50 mg/mL)

| | | | | | Polysorbate | | Ionic |
|---|---|---|---|---|---|---|---|
| | TRIS | Methionine | Trehalose | Mannitol | 20 | | strength* |
| Formulation | (mM) | (mM) | (mM) | (mM) | (mg/mL) | pH | (mM) |
| 2-01 | 1 | — | 300 | — | 0.1 | 7.0 | 0.9 |
| 2-02 | 1 | — | 300 | — | 0.1 | 7.5 | 0.8 |
| 2-03 | 1 | 2 | 150 | 250 | 0.1 | 7.0 | 0.9 |
| 2-04 | 1 | 2 | 150 | 250 | 0.1 | 7.2 | 0.9 |
| 2-05 | 1 | 2 | 150 | 250 | 0.1 | 7.5 | 0.8 |
| 2-06 | 1 | 2 | 150 | 250 | 0.1 | 7.8 | 0.7 |

*Total ionic strength “I” as defined above

Formulations were stored at 25° C. for 26 weeks, or at 2-8° C. for 26 weeks. Stability of PROTEIN-1 was assessed by monitoring the rate of high molecular weight species formation using SEC (Method 1), and by visual assessment, as described in the General Methods.

The rate of HMWS formation in formulations 2-01 to 2-06 is shown in Table 4, where it can be seen that at 25° C. the rate of HMWS formation was lower in a formulation at pH 7.5 compared with a formulation at pH 7.0 at both 25° C. and 2-8° C. (comparing formulations 2-01 and 2-02 with trehalose as uncharged tonicity modifier). Comparing formulations 2-03 to 2-06 (with a mixture of trehalose and mannitol as uncharged tonicity modifier) it can be seen that although again the rate of HMWS formation was lower at pH 7.5 compared with pH 7.0 (for both 25° C. and 2-8° C.), the lowest rate of HMWS formation was observed at pH 7.2.

In summary, pH 7.2-7.5 appears to be particularly suitable for preventing HMWS formation.

TABLE 4
Stability of PROTEIN-1 (50 mg/ml) at 25° C. and 2-8° C.
for 26 weeks in formulations 2-01 to 2-06 assessed
by SEC (Method 1).

| | Increase in % HMWS | |
|---|---|---|
| Formulation | 25° C. | 2-8° C. |
| 2-01 | 3.14 | 1.03 |
| 2-02 | 2.59 | 0.71 |
| 2-03 | 3.04 | 0.90 |
| 2-04 | 2.35 | 0.51 |
| 2-05 | 2.42 | 0.56 |
| 2-06 | 2.86 | 0.71 |

### Example 4—Effect of Buffer Concentration on Storage Stability of Protein-1

The effect of buffer concentration on the stability of PROTEIN-1 (50 mg/ml) was investigated. All formulations contained polysorbate 20 (0.1 mg/ml). Table 5 summarises the formulations tested.

TABLE 5
Formulations of PROTEIN-1 tested. All formulations contained
PROTEIN-1 (50 mg/ml) and polysorbate 20 (0.1 mg/ml)

| | | Sodium | | | | | | Ionic |
|---|---|---|---|---|---|---|---|---|
| | TRIS | phosphate | Methionine | Glycerol | Trehalose | Mannitol | | strength* |
| Formulation | (mM) | (mM) | (mM) | (mM) | (mM) | (mM) | pH | (mM) |
| 3-01 | 50 | — | — | — | — | — | 8.0 | 27.8 |
| 3-02 | 2 | — | — | — | — | — | 8.0 | 1.1 |
| 3-03 | — | 2 | — | 300 | — | — | 7.0 | 3.8 |
| 3-04 | 50 | 2 | — | 300 | — | — | 7.0 | 50.1 |
| 3-05 | 1 | — | 2 | — | 150 | 250 | 7.5 | 0.8 |
| 3-06 | 2 | — | 2 | — | 150 | 250 | 7.5 | 1.6 |
| 3-07 | 5 | — | 2 | — | 150 | 250 | 7.5 | 4 |

*Total ionic strength “I” as defined above

Formulations were stored at 25° C. for 7 weeks and for 27 weeks, or at 2-8° C. for 27 weeks. Stability of PROTEIN-1 was assessed by monitoring the rate of high molecular weight species formation using SEC (Method 1), and by visual assessment, as described in the General Methods.

The rate of HMWS formation in formulations 3-01 to 3-07 is shown in Table 6, where it can be seen that after 7 weeks at 25° C. the rate of HMWS formation was lower in a formulation containing 2 mM TRIS buffer compared with the corresponding formulation containing 50 mM TRIS buffer (comparing formulations 3-01 and 3-02, at pH 8). The same trend was observed at pH 7.0: the rate of HMWS was lower in a formulation containing no TRIS buffer, compared with the corresponding formulation containing 50 mM TRIS buffer (comparing formulations 3-04 and 3-03, both of which contained glycerol as uncharged tonicity modifier, at pH 7.0). The same trend was observed after 27 weeks at both 25° C. and 2-8° C., where reducing the concentration of TRIS buffer from 5 mM, to 2 mM and then to 1 mM led to a reduction in the rate of formation of HMWS (comparing formulations 3-05 to 3-07, which all contained a mixture of trehalose and mannitol as uncharged tonicity modifier, at pH 7.5).

In summary, when using TRIS as buffer for the pH range 7.0-7.5, the concentration should be lower than 50 mM and suitably as low as possible to reduce the formation of HMWS.

TABLE 6
Stability of PROTEIN-1 (50 mg/ml) at 25° C. and 2-8° C.
in formulations 3-01 to 3-07 assessed by SEC (Method 1).

| | Increase in % HMWS | | |
|---|---|---|---|
| | 7 weeks | 27 weeks | |
| Formulation | 25° C. | 25° C. | 2-8° C. |
| 3-01 | 4.14 | | |
| 3-02 | 3.32 | | |
| 3-03 | 1.70 | | |
| 3-04 | 2.84 | | |
| 3-05 | | 2.42 | 0.56 |
| 3-06 | | 2.69 | 0.68 |
| 3-07 | | 2.76 | 0.69 |

### Example 5—Effect of Uncharged Tonicity Modifier on Storage Stability of Protein-1

The effect of different uncharged tonicity modifiers on the stability of PROTEIN-1 (50 mg/ml) was investigated. All formulations contained polysorbate 20 (0.1 mg/ml). Table 7 summarizes the formulations tested.

TABLE 7
Formulations of PROTEIN-1 tested. All formulations contained
PROTEIN-1 (50 mg/ml) and polysorbate 20 (0.1 mg/ml).

| | | | | | | Ionic |
|---|---|---|---|---|---|---|
| | TRIS | Glycerol | Trehalose | Sucrose | | strength* |
| Formulation | (mM) | (mM) | (mM) | (mM) | pH | (mM) |
| 4-01 | 2 | 300 | — | — | 7.0 | 1.9 |
| 4-02 | 2 | — | 300 | — | 7.0 | 1.9 |
| 4-03 | 2 | — | — | 300 | 7.0 | 1.9 |
| 4-04 | 1 | — | 300 | — | 7.5 | 0.8 |
| 4-05 | 1 | — | 200 | — | 7.5 | 0.8 |
| 4-06 | 1 | — | — | 300 | 7.5 | 0.8 |
| 4-07 | 1 | — | — | 200 | 7.5 | 0.8 |

*Total ionic strength “I” as defined above

Formulations were stored at 25° C. for 12 weeks, at 25° C. for 26 weeks, 2-8° C. for 16 weeks or at 2-8° C. for 26 weeks. Stability of PROTEIN-1 was assessed by monitoring the rate of high molecular weight species formation using SEC (Method 1), and by visual assessment, as described in the General Methods.

The rate of HMWS formation in formulations 4-01 to 4-07 is shown in Table 8, where it can be seen that after 12 weeks at 25° C. and after 16 weeks at 2-8° C., comparing the use of glycerol, trehalose and sucrose as uncharged tonicity modifier, the rate of HMWS formation was lowest in the formulation containing trehalose (comparing formulations 4-01 to 4-03, at pH 7, with 2 mM TRIS buffer). The same was observed for longer storage periods of 26 weeks, comparing the use of trehalose and sucrose as uncharged tonicity modifier, the rate of HMWS was lower in the formulation containing trehalose (comparing formulations 4-04 to 4-07, at pH 7.5, with 1 mM TRIS buffer). Of the two trehalose concentrations tested (200 mM and 300 mM), a lower rate of HMWS formation was observed at the higher concentration of 300 mM (comparing formulations 4-04 and 4-05). However, in some cases, particularly if the dose volume of the composition is high, the concentration of trehalose may be limited due to pharmacologically acceptable limits, for example to around 150 mM. In such cases a mixture of trehalose with another uncharged tonicity modifier, for example sucrose, may be optimal.

In summary, using trehalose as uncharged tonicity modifier provided the best stability profile for the concentrations and tonicity modifiers tested.

TABLE 8
Stability of PROTEIN-1 (50 mg/ml) at 25° C. and 2-8° C.
in formulations 4-01 to 4-07 assessed by SEC (Method 1).

| | Increase in % HMWS | | | |
|---|---|---|---|---|
| | 25° C. | 25° C. | 2-8° C. | 2-8° C. |
| Formulation | (12 weeks) | (26 weeks) | (16 weeks) | (26 weeks) |
| 4-01 | 2.90 | | 3.19 | |
| 4-02 | 1.43 | | 0.50 | |
| 4-03 | 1.65 | | 0.85 | |
| 4-04 | | 2.59 | | 0.71 |
| 4-05 | | 3.77 | | 2.19 |
| 4-06 | | 3.21 | | 1.37 |
| 4-07 | | 4.27 | | 2.93 |

### Example 6—Effect of Neutral Amino Acids on Storage Stability of Protein-1

The effect of different neutral amino acids on the stability of PROTEIN-1 (50 mg/ml) was investigated. All formulations contained polysorbate 20 (0.1 mg/ml), TRIS buffer (2 mM), and glycerol (300 mM) as uncharged tonicity modifier. Table 9 summarises the formulations tested.

TABLE 9
Formulations of PROTEIN-1 tested. All formulations contained
PROTEIN-1 (50 mg/mL) and polysorbate 20 (0.1 mg/ml).

| | | | | | | Ionic |
|---|---|---|---|---|---|---|
| | TRIS | Glycerol | Glycine | Proline | | strength* |
| Formulation | (mM) | (mM) | (mM) | (mM) | pH | (mM) |
| 5-01 | 2 | 300 | 100 | — | 7.0 | 1.9 |
| 5-02 | 2 | 300 | 300 | — | 7.0 | 1.9 |
| 5-03 | 2 | 300 | — | 100 | 7.0 | 1.9 |
| 5-04 | 2 | 300 | — | 300 | 7.0 | 1.9 |
| 5-05 | 2 | 300 | — | 500 | 7.0 | 1.9 |
| 5-06 | 2 | 300 | — | — | 7.0 | 1.9 |

*Total ionic strength “I” as defined above

Formulations were stored at 25° C. for 16 weeks. Stability of PROTEIN-1 was assessed by monitoring the rate of high molecular weight species formation using SEC (Method 1), and by visual assessment, as described in the General Methods.

The rate of HMWS formation in formulations 5-01 to 5-06 is shown in Table 10, where it can be seen that after 16 weeks at 25° C., the addition of both glycine (at 300 mM) and proline (at all concentrations tested) resulted in a lower rate of HMWS formation (comparing formulations 5-01 to 5-05 with control formulation 5-06). Comparing glycine and proline directly, at a given concentration of neutral amino acid, the rate of HMWS formation was lower in the formulations containing proline (comparing formulation 5-01 with formulation 5-03, and comparing formulation 5-02 with formulation 5-04). Of the three proline concentrations tested (100 mM, 200 mM and 500 mM), the lowest rate of HMWS formation was observed at the highest concentration of 500 mM (comparing formulations 5-05 and 5-05). However, in practice the concentration of proline used in a commercial therapeutic formulation would ideally be lower (e.g. around 100-150 mM) due to osmolarity limitations.

In summary, the addition of a neutral amino acid (e.g. proline or glycine, particularly proline) resulted in a more stable formulation.

TABLE 10
Stability of PROTEIN-1 (50 mg/ml) at 25° C. for 16 weeks in
formulations 5-01 to 5-06 assessed by SEC (Method 1).

| Formulation | Increase in % HMWS |
|---|---|
| 5-01 | 3.96 |
| 5-02 | 3.18 |
| 5-03 | 3.57 |
| 5-04 | 2.88 |
| 5-05 | 2.40 |
| 5-06 | 3.82 |

### Example 7—Effect of Methionine in Combination with Another Neutral Amino Acid, on Storage Stability of Protein-1

The effect of methionine in combination with either of neutral amino acids glycine or proline, on the stability of PROTEIN-1 (50 mg/ml) was investigated. All formulations contained polysorbate 20 (0.1 mg/ml), TRIS buffer (2 mM or 1 mM), and glycerol (300 mM) or trehalose (300 mM) as uncharged tonicity modifier. Table 11 summarizes the formulations tested.

TABLE 11
Formulations of PROTEIN-1 tested. All formulations contained
PROTEIN-1 (50 mg/ml) and polysorbate 20 (0.1 mg/ml).

| | | | | | | | | Ionic |
|---|---|---|---|---|---|---|---|---|
| | TRIS | Methionine | Glycerol | Glycine | Proline | Trehalose | | strength* |
| Formulation | (mM) | (mM) | (mM) | (mM) | (mM) | (mM) | pH | (mM) |
| 6-01 | 2 | — | 300 | 300 | — | — | 7.0 | 1.9 |
| 6-02 | 2 | 10 | 300 | 300 | — | — | 7.0 | 1.9 |
| 6-03 | 1 | — | — | — | 200 | 300 | 7.5 | 0.8 |
| 6-04 | 1 | 2 | — | — | 200 | 300 | 7.5 | 0.8 |

*Total ionic strength “I” as defined above

Formulations were stored at 25° C. for 16 weeks, at 25° C. for 26 weeks, or at 2-8° C. for 26 weeks. Stability of PROTEIN-1 was assessed by monitoring the rate of high molecular weight species formation using SEC (Method 1), and by visual assessment, as described in the General Methods.

The rate of HMWS formation in formulations 6-01 to 6-04 is shown in Table 12, where it can be seen that after 16 weeks at 25° C., the addition of methionine to a formulation containing glycine led to a lower rate of formation of HMWS (comparing formulations 6-01 and 6-02). The addition of methionine to a formulation containing proline also led to a lower rate of formation of HMWS after 26 weeks at 25° C. or after 26 weeks at 2-8° C. (comparing formulations 6-03 and 6-04).

TABLE 12
Stability of PROTEIN-1 (50 mg/ml) at 25° C. in formulations
6-01 to 6-04 assessed by SEC (Method 1).

| | Increase in % HMWS | | |
|---|---|---|---|
| | 25° C. | 25° C. | 2-8° C. |
| Formulation | (16 weeks) | (26 weeks) | (26 weeks) |
| 6-01 | 3.18 | | |
| 6-02 | 2.62 | | |
| 6-03 | | 2.15 | 0.15 |
| 6-04 | | 1.87 | 0.07 |

### Example 8—Storage Stability Testing of Formulation of Protein-1 with Poloxamer Surfactant

The effect of poloxamer surfactant on the stability of PROTEIN-1 (50 mg/ml) was investigated. The batch of PROTEIN-1 used in this example was a different batch to that used in Examples 2-7. Table 13 summarises the formulation tested.

TABLE 13
Formulations of PROTEIN-1 tested. The formulation contained PROTEIN-1 (50 mg/ml).

| | | | | | | Poloxamer | | Ionic |
|---|---|---|---|---|---|---|---|---|
| | TRIS | Methionine | Trehalose | Sucrose | Proline | 188 | | strength* |
| Formulation | (mM) | (mM) | (mM) | (mM) | (mM) | (mg/ml) | pH | (mM) |
| 7-01 | 2 | 2 | 150 | 100 | 100 | 1.0 | 7.5 | 1.6 |

*Total ionic strength “I” as defined above

The formulation was stored at 25° C. and at 2-8° C. for 6 months, 9 months, 12 months and 24 months. Stability of PROTEIN-1 was assessed by monitoring the rate of high molecular weight species formation using SEC (Method 1), and by visual assessment, as described in the General Methods.

The formulation had a low rate of HMWS formation under the storage conditions as can be seen from Tables 14A and 14B.

As shown in Tables 15A and 15B, the formulation tested passed the visual test following storage at 25° C. and at 2-8° C.

TABLE 14A
Stability of PROTEIN-1 (50 mg/ml) at 2-8° C. in formulation
7-01 assessed by SEC (Method 1).

| | Increase in % HMWS at 2-8° C. | | | | |
|---|---|---|---|---|---|
| | 6 | 9 | 12 | 18 | 24 |
| Formulation | months | months | months | months | months |
| 7-01 | 1.08 | 1.71 | 2.51 | 4.03 | 5.61 |

TABLE 14B
Stability of PROTEIN-1 (50 mg/ml) at 25° C. in formulation
7-01 assessed by SEC (Method 1).

| | Increase in % HMWS at 25° C. | | | | |
|---|---|---|---|---|---|
| | 6 | 9 | 12 | 18 | 24 |
| Formulation | months | months | months | months | months |
| 7-01 | 3.36 | 5.06 | 7.19 | 11.10 | 15.49 |

TABLE 15A
Stability of PROTEIN-1 (50 mg/ml) at 2-8° C. in formulation
7-01 assessed using visual assessment.

| | Visual score at 2-8° C. | | | | |
|---|---|---|---|---|---|
| | 6 | 9 | 12 | 18 | 24 |
| Formulation | months | months | months | months | months |
| 7-01 | 1 | 1 | 1 | 1 | 1 |

TABLE 15B
Stability of PROTEIN-1 (50 mg/ml) at 25° C. in formulation 7-01
assessed using visual assessment.

| | Visual score at 25° C. | | | | |
|---|---|---|---|---|---|
| Formulation | 6 months | 9 months | 12 months | 18 months | 24 months |
| 7-01 | 1 | 1 | 1 | 1 | 1 |

### Example 9—Sub Visible Particle Size Limit Assessment for Formulation of Protein-1

The stability of the formulation of PROTEIN-1 set out in Table 16A was assessed by visual assessment, and by determining the number of sub visible particles (SVP) after storage for 6 months at 25° C., both as described in the General Methods.

TABLE 16A
Formulation of PROTEIN-1 (50 mg/ml) tested.

| | | | | | | Poloxamer | | Ionic |
|---|---|---|---|---|---|---|---|---|
| | TRIS | Methionine | Trehalose | Sucrose | Proline | 188 | | strength* |
| Formulation | (mM) | (mM) | (mM) | (mM) | (mM) | (mg/ml) | pH | (mM) |
| 9-01 | 5 | 2 | 150 | 100 | 100 | 1.0 | 7.3 | 4.3 |

*Total ionic strength “I” as defined above

Following storage for 6 months at 25° C., Formulation 9-01 was practically free of visible particles. The number of sub visible particles is shown in Table 16B.

TABLE 16B
Number of sub visible particles following storage of Formulation
9-01 for 6 months at 25° C.

| | Number of sub visible particles per container | |
|---|---|---|
| Formulation | ≤10 μm | ≤25 μm |
| 9-01 | 67 | 0 |

USP <788>“Particulate matter in injections” 11 (harmonized with Ph. Eur. 2.9.19) requires preparations of less than 100 mL to contain no more than 6000 particles ≥10 μm per container and no more than 600 particles ≥25 μm per container as determined by light obscuration (such as the HIAC method in the General Methods). As such, following long term storage Formulation 9-01 is well below the necessary upper limits for sub visible particles, indicating excellent storage stability.

### Example 10—Design of Experiment (DOE) Study Assessing Concentration of Formulation Components on Stability of Protein-1

The effect of varying concentrations of protein, buffer and poloxamer surfactant on the stability of PROTEIN-1 was assessed by monitoring the rate of formation of high molecular weight species in the formulations using SEC (Method 2), as described in the General Methods. The rate of HMWS formation in in the various formulations after 12 months at 5° C. is shown in Table 17.

TABLE 17
Stability of PROTEIN-1 at 5° C. for 12 months assessed by SEC
(Method 2).

| | PROTEIN-1 | Tris | | Poloxamer 188 | Increase in % |
|---|---|---|---|---|---|
| Formulation | (mg/mL) | (mM) | pH | (%) | HMWS @ 12 m |
| RF08 | 58 | 10 | 7.7 | 0.03 | 3.3 |
| RF01 | 58 | 10 | 7.7 | 0.17 | 3.3 |
| RF15 | 58 | 2 | 7.7 | 0.03 | 2.6 |
| RF16 | 58 | 2 | 7.7 | 0.17 | 2.4 |
| RF02 | 42 | 10 | 7.7 | 0.03 | 2.4 |
| RF11 | 42 | 10 | 7.7 | 0.17 | 2.4 |
| RF09 | 42 | 2 | 7.7 | 0.03 | 1.7 |
| RF05 | 42 | 2 | 7.7 | 0.17 | 1.7 |

All formulations tested exhibited good storage stability. Comparing Formulations RF08 with RF02, RF01 with RF11, RF15 with RF09, and RF16 with RF05, it can be seen that lowering the protein concentration results in a slight improvement in stability. Comparing Formulations RF08 and RF01 with RF15 and RF16, and comparing Formulations RF02 and RF11 with RF09 and RF05, it can be seen that a lower buffer (TRIS) concentration results in a slight improvement in stability, although the higher buffer (TRIS) concentration of 10 mM still provides a formulation with good stability. Finally, the effect on storage stability of raising the poloxamer concentration from 0.03% to 0.17% was minimal.

### Comparative Example 11—Effect of Buffer Concentration, Charge of Tonicity Modifier and a Neutral Amino Acid on Storage Stability of an Immunoglobulin G1 (IgG1) at 30° C.

The effect of buffer concentration and charge of the tonicity modifier on stability of an IgG1 (100 mg/ml) was investigated. Citrate buffer was tested. Sodium chloride (150 mM) was used as a charged tonicity modifier and glycerol (300 mM) was used as an uncharged tonicity modifier. The effect of proline and glycine (50 mM) on stability of the IgG1 was also investigated in the presence of 1 mM buffer and glycerol (300 mM). All formulations tested contained polysorbate 80 (0.2 mg/ml) and were adjusted to pH 6.0. Table 18 summarizes the formulations tested. All formulations were stressed at 30° C. for 8 weeks. Stability of the IgG1 was followed by monitoring the rate of high molecular weight species formation using SEC (Method 1).

TABLE 18
Formulations of IgG1 tested. All formulations contained
IgG1 (100 mg/ml) and polysorbate 80 (0.2 mg/ml) and were
adjusted to pH 6.0.

| | Citric | Sodium | | | | Ionic |
|---|---|---|---|---|---|---|
| | acid | chloride | Glycerol | Proline | Glycine | strength* |
| Formulation | (mM) | (mM) | (mM) | (mM) | (mM) | (mM) |
| 8-01 | 1 | 150 | — | — | — | 153.8 |
| 8-02 | 5 | 150 | — | — | — | 168.7 |
| 8-03 | 20 | 150 | — | — | — | 224.8 |
| 8-04 | 1 | — | 300 | — | — | 3.8 |
| 8-05 | 5 | — | 300 | — | — | 18.7 |
| 8-06 | 20 | — | 300 | — | — | 74.8 |
| 8-07 | 1 | — | 300 | 50 | — | 3.8 |
| 8-08 | 1 | — | 300 | — | 50 | 3.8 |

*Total ionic strength “I” as defined above

All formulations tested passed the visual test following storage at 30° C. The rate of HMWS formation in formulations 8-01 to 8-08 following storage at 30° C. is shown in Table 19. The rate of HMWS formation decreased with increasing buffer concentration both in the presence of sodium chloride and in the presence of glycerol (comparing formulations 8-01 to 8-03, and comparing formulations 8-04 to 8-06). There was a slight trend for higher stability at higher ionic strength of the formulation when the citric acid concentration was low (1 or 5 mM) (comparing formulation 8-01 with formulation 8-04 and comparing formulation 8-02 with formulation 8-05). This order was reversed when the citric acid concentration was high (20 mM) (comparing formulation 8-03 with formulation 8-06) although in this instance the ionic strength of both formulations was above 70 mM. The presence of a neutral amino acid (proline or glycine) resulted in a very slight increase in the rate of HMWS formation (comparing formulation 8-04 with formulations 8-07 and 8-08).

TABLE 19
Stability of IgG1 (100 mg/ml) at 30° C. in formulations 8-01 to 8-08
assessed by SEC (Method 1).

| | Increase in % HMWS following incubation at |
|---|---|
| Formulation | 30° C. for 8 weeks |
| 8-01 | 6.28 |
| 8-02 | 5.15 |
| 8-03 | 4.41 |
| 8-04 | 6.58 |
| 8-05 | 5.48 |
| 8-06 | 4.27 |
| 8-07 | 6.67 |
| 8-08 | 6.68 |

### Summary of the Examples

The data of Examples 2-10 shows that compositions of the engineered dimeric protein of the invention as defined herein such as PROTEIN-1 are stable when formulated at low ionic strength with a neutral amino acid. The ionic strength of a composition is suitably kept low by using a low buffer concentration (or by not using any buffer) and by using an uncharged tonicity modifier instead of a charged tonicity modifier.

When these data are compared to Comparative Example 11, it can be seen that this behavior is quite different to the behavior exhibited by the tested 4-chain antibody (type IgG1). The latter was found to be more stable at higher buffer concentrations and, in some cases, at higher ionic strength and was also found to be destabilized in the presence of a neutral amino acid.

The present invention combines composition features that, without being limited by theory, are believed to work in concert to screen unnatural hydrophobic patches as well as minimizing the rate of proton exchange at unnaturally exposed sites of instability, resulting in stability of the engineered dimeric protein of the invention such as PROTEIN-1.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All patents, patent applications and references mentioned throughout the specification of the present invention are herein incorporated in their entirety by reference.

The invention embraces all combinations of preferred and more preferred groups and suitable and more suitable groups and embodiments of groups recited above.

1. An aqueous solution composition of pH in the range 6.0 to 8.0 comprising:
an engineered dimeric protein wherein each monomer of the dimeric protein comprises at least one human serpin polypeptide operably linked to a human immunoglobulin Fc polypeptide or a polypeptide that is derived from an immunoglobulin Fc polypeptide;
optionally one or more buffers being substances having at least one ionisable group with a pKa in the range 4.0 to 10.0 and which pKa is within 2 pH units of the pH of the composition;
a neutral amino acid; and
an uncharged tonicity modifier;
wherein the buffers are present in the composition at a total concentration of 0-10 mM; and
wherein the total ionic strength of the composition excluding the contribution of the engineered dimeric protein is less than 30 mM.

2. The aqueous solution composition of claim 1, wherein the human serpin polypeptide is a human alpha-1 antitrypsin (AAT) polypeptide or is derived from a human AAT polypeptide.

3. The aqueous solution composition of claim 1, wherein each monomer of the dimeric protein comprises one human serpin polypeptide.

4. The aqueous solution composition of claim 2, wherein the human serpin polypeptide has the sequence of SEQ ID NO: 1 or 2.

5. The aqueous solution composition of claim 1, wherein the human immunoglobulin Fc polypeptide or a polypeptide that is derived from an immunoglobulin Fc polypeptide is a modified human IgG4 Fc polypeptide.

6. The aqueous solution composition of claim 5, wherein the human immunoglobulin Fc polypeptide or a polypeptide that is derived from an immunoglobulin Fc polypeptide is a modified human IgG4 Fc polypeptide and has the sequence of any one of SEQ ID NOs: 28-43.

7. The aqueous solution composition of claim 6, wherein each monomer of the dimeric protein has the sequence of SEQ ID No: 56.

8. The aqueous solution composition of claim 1, wherein the protein is present at a concentration of 1-400 mg/ml.

9. The aqueous solution composition of claim 1, wherein buffers are present at a total concentration of 0.1-10 mM.

10. The aqueous solution composition of claim 1, which is substantially free of buffers.

11. The aqueous solution composition of claim 1, wherein the buffer comprises ionisable groups with pKa within 1 unit of the pH of the composition.

12. The aqueous solution composition of claim1, wherein the buffer or buffers is/are selected from the group consisting of citrate, histidine, maleate, sulphite, aspartame, aspartate, glutamate, tartrate, adenine, succinate, ascorbate, benzoate, phenylacetate, gallate, cytosine, p-aminobenzoic acid, sorbate, acetate, propionate, alginate, urate, 2-(N-morpholino)ethanesulphonic acid, bicarbonate, bis(2-hydroxyethyl) iminotris(hydroxymethyl)methane, N-(2-acetamido)-2-iminodiacetic acid, 2-[(2-amino-2-oxoethyl)amino]ethanesulphonic acid, piperazine, N,N′-bis(2-ethanesulphonic acid), phosphate, N,N-bis(2-hydroxyethyl)-2-aminoethanesulphonic acid, 3-[N,N-bis(2-hydroxyethyl)amino]-2-hydroxypropanesulphonic acid, triethanolamine, piperazine-N,N′-bis(2-hydroxypropanesulphonic acid), tris(hydroxymethyl)aminomethane (TRIS), N tris(hydroxymethyl)glycine and N-tris(hydroxymethyl)methyl-3-aminopropanesulphonic acid, and salts thereof, and combinations thereof.

13. The aqueous solution composition of claim 1, wherein the uncharged tonicity modifier is selected from the group consisting of polyols, sugars and sugar alcohols, wherein the sugars is monosaccharides or disaccharides.

14. The aqueous solution composition of claim 13, wherein the uncharged tonicity modifier is selected from the group consisting of glycerol, 1,2-propanediol, mannitol, sorbitol, glucose, sucrose, trehalose, PEG300 and PEG400or a combination thereof.

15. The aqueous solution composition of claim 1, wherein the total concentration of the one or more uncharged tonicity modifier is 50-1000 mM.

16. The aqueous solution composition of claim 1, wherein the osmolarity of the composition is 200-500 mOsm/L.

17. The aqueous solution composition of claim 1, comprising a neutral amino acid selected from glycine, methionine, proline, alanine, valine, leucine, isoleucine, phenylalanine, tyrosine, tryptophan, serine, threonine, asparagine and glutamine or a combination thereof, wherein the total concentration of the one or more neutral amino acids in the composition is 20 to 600 mM.

18. The aqueous solution composition of claim 1, comprises a non-ionic surfactant selected from the group consisting of an alkyl glycoside, a polysorbate, an alkyl ether of polyethylene glycol, a block copolymer of polyethylene glycol and polypropylene glycol (poloxamer), and an alkylphenyl ether of polyethylene glycol, wherein the non-ionic surfactant is present at a concentration of 10-2000 μg/ml.

19. The aqueous solution composition of claim 1, which comprises a preservative, wherein the preservative is a phenolic or benzylic preservative, and wherein the phenolic or benzylic preservative is selected from the group consisting of phenol, m-cresol, chlorocresol, benzyl alcohol, propyl paraben and methyl paraben, and wherein the preservative is present at a concentration of 10-100 mM.

20. A method of inhibiting or downregulating aberrant serine protease expression or activity in a subject in need thereof, the method comprising administering an aqueous solution composition of claim 1.
